(19) United States
(12) Patent Application Publication
(10) Pub. No.: US 2002/0098174 A1
(43) Pub. Date: Jul. 25, 2002

(54) 33166, a human hydrolase-like molecule and uses thereof

(76) Inventors: Rosana Kapeller-Libermann, Chestnut Hill, MA (US); Kyle J. MacBeth, Boston, MA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/775,117

(22) Filed: Feb. 1, 2001

Related U.S. Application Data
(60) Provisional application No. 60/194,065, filed on Mar. 31, 2000.

(51) Int. Cl. A61K 38/46; C12N 9/16; C07H 21/04; C12P 21/02; C12N 5/06

(52) U.S. Cl. 424/94.6; 435/69.1; 435/196; 435/320.1; 435/325; 536/23.2

(57) ABSTRACT

Novel alpha/beta hydrolase-like polypeptides, proteins, and nucleic acid molecules are disclosed. In addition to isolated, full-length alpha/beta hydrolase-like proteins, the invention further provides isolated alpha/beta hydrolase-like fusion proteins, antigenic peptides, and anti-alpha/beta hydrolase-like antibodies. The invention also provides alpha/beta hydrolase-like nucleic acid molecules, recombinant expression vectors containing a nucleic acid molecule of the invention, host cells into which the expression vectors have been introduced, and nonhuman transgenic animals in which an alpha/beta hydrolase-like gene has been introduced or disrupted. Diagnostic, screening, and therapeutic methods utilizing compositions of the invention are also provided. Therapeutic methods for treating breast, lung, colon, brain, and ovary cancers using the hydrolase-like molecules are described.

### CROSS-REFERENCE TO RELATED APPLICATION

This application claims the benefit of U.S. Provisional No. 60/194,065 filed Mar. 31, 2000.

### FIELD OF THE INVENTION

The invention relates to novel alpha/beta hydrolase-like nucleic acid sequences and proteins. Also provided are vectors, host cells, and recombinant methods for making and using the novel molecules.

### BACKGROUND OF THE INVENTION

The alpha/beta hydrolase (ABH) fold family of proteins encompasses members with diverse phylogenetic origin and function. The majority of the ABH fold proteins are hydrolytic enzymes catalyzing hydrolysis of a wide variety of bonds including ester, amide, epoxide, C-halogen, and even C—C bonds. Enzyme members include lipases, esterases, proteases, and various other enzymes. Nonenzyme proteins in this family include proteins such as glutactin, vitellogenin, thyroglobulin, and neuroligin. (Fischer et al. (1999) Journal of Bacteriology 181(18): 5725-5733; Zhang, et al, (1998) Folding & Design 3(6): 535-548).

Lipase members of the ABH family include hepatic-, glycerol-, bacterial-, pancreatic, lipoprotein- and hormone sensitive lipases. Esterase members include cutinase, thioesterase, carboxylesterase, cholesterol esterase, acetylcholinesterase, and butyrylcholinesterase. Protease members include carboxypeptidase and prolyl aminopeptidase. Other enymes in this family include bacterial 2,4-dioxygenases, bromoperoxidase, hydroxynitrile lyase, sterol acyltransferase, hydrolase, haloalkane dehalogenase (Morel, et al. (1999) Biochimica et Biophysica Acta—Protein & Molecular Enzymology 1429(2): 501-505; Fischer et al., 1999, Journal of Bacteriology 181(18): 5725-5733; Zhang, et al. (1998) Folding & Design 3(6): 535-548).

The involvement of lipases in lipid and cholesterol metabolism is well known. Likewise, the involvement of serine hydolases such as carboxylesterase, cholesterol esterase, acetylcholinesterase, and butyrylcholinesterase in pharmacology and toxicology are well known. For example, acetylcholinesterase inhibitors are useful as insecticides due to their toxic effects and as therapeutic agents for treatment of Alzheimer's disease, myasthenia gravis and glaucoma. Another member of the ABH superfamily with recognized pharmacological significance is epoxide hydrolase which is involved in detoxification of highly harmful aromatic compounds in mammals. The human hormone sensitive lipase performs the important rate-limiting step of hydrolysing fat stored in adipocytes. See, for example Heikinheimo et al (1999) Structure. 7(6): R141-R146; Satoh and Hosokawa (1995), Toxicol Lett: 439-45.

The ABH fold family was initially identified by comparing several divergent hydrolytic enzymes having a core topology of eight beta-sheets connected by alpha-helices, and a conserved catalytic triad (Ollis et al. (1992) Protein Eng 5(3): 197-211). With the growth of the family, the topology has been expanded to encompasses other variations. Nevertheless, the catalytic triad of nucleophilic-, acidic-, and histidine residues remains a common feature among the enzyme members of the family. For example, Heikinheimo et al. (1999) Structure 7(6): R141-R146, describe nine variations of the ABH fold structures, in addition to a canonical and minimal structure; all having the catalytic triad residues. Within the catalytic triad, the nucleophile residue has included serine, cysteine or aspartate; and the acid residue has included glutamate. Further information on structural and functional aspects of ABH fold proteins are available, for example, as described by Zhang et al., (1998) Folding & Design 3(6): 535-548;

Due to the diversity of the ABH fold family, members of this family are implicated in numerous cellular, physiological, and pathological processes. Such processes include lipid and cholesterol metabolism; biotransformation of drugs and other chemicals; detoxification; neurotransmission; and cellular cycle regulation, growth and differentiation. Thus, methods and compositions are needed for modulating these processes.

### SUMMARY OF THE INVENTION

Isolated nucleic acid molecules corresponding to alpha/beta hydrolase-like nucleic acid sequences are provided. Additionally, amino acid sequences corresponding to the polynucleotides are encompassed. In particular, the present invention provides for isolated nucleic acid molecules comprising nucleotide sequences encoding the amino acid sequences shown in SEQ ID NO:2 or the nucleotide sequences encoding the DNA sequence deposited in a bacterial host as ATCC Accession Number ______. Further provided are alpha/beta hydrolase-like polypeptides having an amino acid sequence encoded by a nucleic acid molecule described herein.

The present invention also provides vectors and host cells for recombinant expression of the nucleic acid molecules described herein, as well as methods of making such vectors and host cells and for using them for production of the polypeptides or peptides of the invention by recombinant techniques.

The alpha/beta hydrolase-like molecules of the present invention are useful for modulating lipid and cholesterol metabolism; biotransformation of drugs and other chemicals; detoxification; neurotransmission; cellular cycle regulation, growth and differentiation. The molecules are useful for the diagnosis and treatment of disorders associated with these processes including, but not limited to hyperproliferative and neurogenerative disorders, and drug-induced toxicities. Accordingly, in one aspect, this invention provides isolated nucleic acid molecules encoding alpha/beta hydrolase-like proteins or biologically active portions thereof, as well as nucleic acid fragments suitable as primers or hybridization probes for the detection of alpha/beta hydrolase-like-encoding nucleic acids.

Another aspect of this invention features isolated or recombinant alpha/beta hydrolase-like proteins and polypeptides. Preferred alpha/beta hydrolase-like proteins and polypeptides possess at least one biological activity possessed by naturally occurring alpha/beta hydrolase-like proteins.

Variant nucleic acid molecules and polypeptides substantially homologous to the nucleotide and amino acid sequences set forth in the sequence listings are encompassed by the present invention. Additionally, fragments and substantially homologous fragments of the nucleotide and amino acid sequences are provided.

Antibodies and antibody fragments that selectively bind the alpha/beta hydrolase-like polypeptides and fragments are provided. Such antibodies are useful in detecting the alpha/beta hydrolase-like polypeptides as well as in regulating lipid and cholesterol metabolism; biotransformation of drugs and other chemicals; detoxification; neurotransmission; cellular cycle regulation, growth and differentiation.

In another aspect, the present invention provides a method for detecting the presence of alpha/beta hydrolase-like activity or expression in a biological sample by contacting the biological sample with an agent capable of detecting an indicator of alpha/beta hydrolase-like activity such that the presence of alpha/beta hydrolase-like activity is detected in the biological sample.

In yet another aspect, the invention provides a method for modulating alpha/beta hydrolase-like activity comprising contacting a cell with an agent that modulates (inhibits or stimulates) alpha/beta hydrolase-like activity or expression such that alpha/beta hydrolase-like activity or expression in the cell is modulated. In one embodiment, the agent is an antibody that specifically binds to alpha/beta hydrolase-like protein. In another embodiment, the agent modulates expression of alpha/beta hydrolase-like protein by modulating transcription of an alpha/beta hydrolase-like gene, splicing of an alpha/beta hydrolase-like mRNA, or translation of an alpha/beta hydrolase-like mRNA. In yet another embodiment, the agent is a nucleic acid molecule having a nucleotide sequence that is antisense to the coding strand of the alpha/beta hydrolase-like mRNA or the alpha/beta hydrolase-like gene.

In one embodiment, the methods of the present invention are used to treat a subject having a disorder characterized by aberrant alpha/beta hydrolase-like protein activity or nucleic acid expression by administering an agent that is an alpha/beta hydrolase-like modulator to the subject. In one embodiment, the alpha/beta hydrolase-like modulator is an alpha/beta hydrolase-like protein. In another embodiment, the alpha/beta hydrolase-like modulator is an alpha/beta hydrolase-like nucleic acid molecule. In other embodiments, the alpha/beta hydrolase-like modulator is a peptide, peptidomimetic, or other small molecule.

The present invention also provides a diagnostic assay for identifying the presence or absence of a genetic lesion or mutation characterized by at least one of the following: (1) aberrant modification or mutation of a gene encoding an alpha/beta hydrolase-like protein; (2) misregulation of a gene encoding an alpha/beta hydrolase-like protein; and (3) aberrant post-translational modification of an alpha/beta hydrolase-like protein, wherein a wild-type form of the gene encodes a protein with an alpha/beta hydrolase-like activity.

In another aspect, the invention provides a method for identifying a compound that binds to or modulates the activity of an alpha/beta hydrolase-like protein. In general, such methods entail measuring a biological activity of an alpha/beta hydrolase-like protein in the presence and absence of a test compound and identifying those compounds that alter the activity of the alpha/beta hydrolase-like protein.

The invention also features methods for identifying a compound that modulates the expression of alpha/beta hydrolase-like genes by measuring the expression of the alpha/beta hydrolase-like sequences in the presence and absence of the compound.

Other features and advantages of the invention will be apparent from the following detailed description and claims.

### BRIEF DESCRIPTION OF THE DRAWINGS

FIG. 1 provides the nucleotide and amino acid sequence for clone 33166.

FIG. 2 shows a hydrophobicity plot of the hydrolase.

FIG. 3 shows an analysis of the hydrolase open reading frame for amino acids corresponding to specific functional sites. N-glycosylation sites are found from about amino acid 108 to 111, and from about amino acid 332 to about amino acid 335. Glycosaminoglycan attachment sites are from about amino acid 138 to 141 and from about amino acid 142 to about 145. cAMP and cGMP-dependent protein kinase phosphorylation sites are from about amino acid 80 to about 83 and from about 164 to about amino acid 167. A protein kinase C phosphorylation site is from about amino acid 168 to about amino acid 170 and from about amino acid 423 to about amino acid 425. A casein kinase II phosphorylation site is from about amino acid 34 to about amino acid 37 and from about amino acid 281 to about amino acid 284. N-myristoylation sites are from about amino acids 4 to 9; 15 to 20; 74 to 79; 106 to 111; 134 to 139; 141 to 146; 183 to 188; 254 to 259; 277 to 282; and 328 to 333. An amidation site is from about amino acid 145 to about amino acid 148.

FIG. 4 shows microarray expression data in a graphical presentation of median-normalized intensity values for clone (Mine 33166) in human breast tissue samples profiled on the 25K array.

FIGS. 5A and 5B show Taqman expression data in clinical tumor samples on the human oncology tissue panel.

FIGS. 6A and 6B show Taqman expression data in clinical tumor samples on human oncology tissue panel.

### DETAILED DESCRIPTION OF THE INVENTION

The present invention provides alpha/beta hydrolase-like molecules. By “alpha/beta hydrolase-like molecules” is intended a novel human sequence referred to as 33166, and variants and fragments thereof. These full-length gene sequences or fragments thereof are referred to as “alpha/beta hydrolase-like” sequences, indicating they share sequence similarity with alpha/beta hydrolase genes. Isolated nucleic acid molecules comprising nucleotide sequences encoding the 33166 polypeptide whose amino acid sequence is given in SEQ ID NO:2, or a variant or fragment thereof, are provided. A nucleotide sequence encoding the 33166 polypeptide is set forth in SEQ ID NO:1. The sequences are members of the ABH fold family of proteins.

A novel human alpha/beta hydrolase-like gene sequence, referred to as 33166, is provided. This gene sequence and variants and fragments thereof are encompassed by the term “alpha/beta hydrolase-like” molecules or sequences as used herein. The alpha/beta hydrolase-like sequences find use in modulating a alpha/beta hydrolase-like function. By “modulating” is intended the upregulating or downregulating of a response. That is, the compositions of the invention affect the targeted activity in either a positive or negative fashion. The sequences of the invention find use in modulating the processes including, but not limited to lipid and cholesterol metabolism; biotransformation of drugs and other chemicals; detoxification; neurotransmission; cellular cycle regulation, growth and differentiation. The disclosed invention relates to methods and compositions for the modulation, diagnosis, and treatment of disorders associated with these processes including, but not limited to hyperproliferative and neurogenerative disorders, and drug-induced toxicities. Examples of such disorders include but are not limited to cancers, Alzheimer's disease, atherosclerosis, and arene oxide-related toxicity. More particularly, cancers of the breast, lung, colon, brain and ovary may be treated with the 33166 gene or variants or fragments thereof. Additionally, a polypeptide comprising the amino acid sequence of SEQ ID NO:2 or a naturally occurring variant or fragment thereof may be used to treat such cancers.

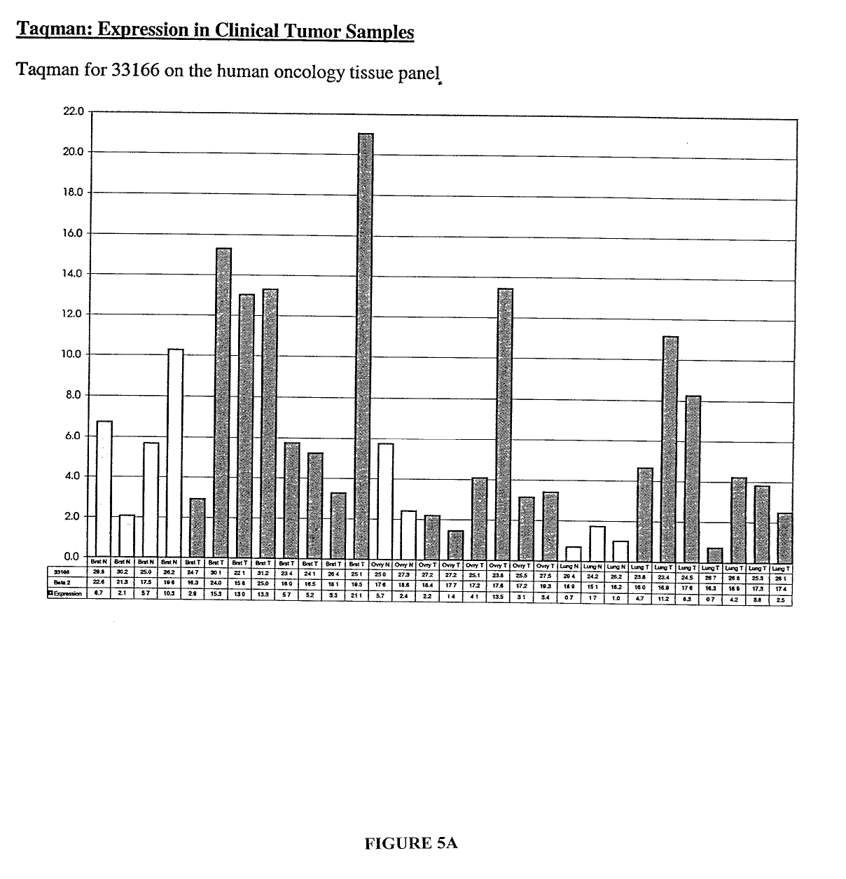

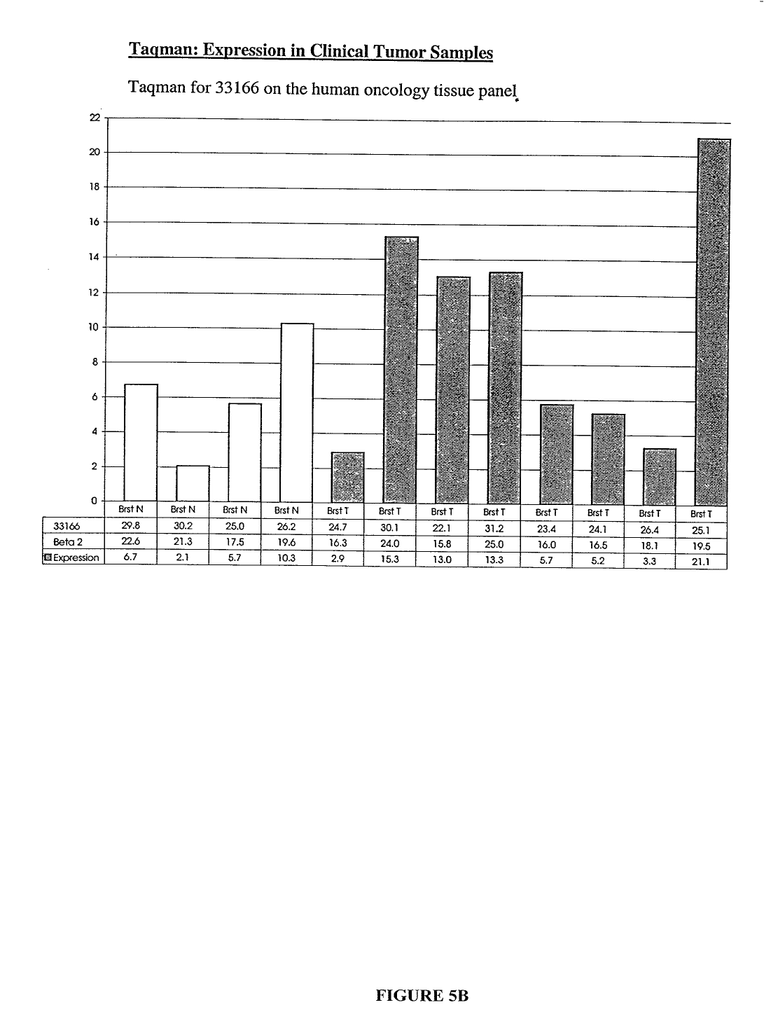

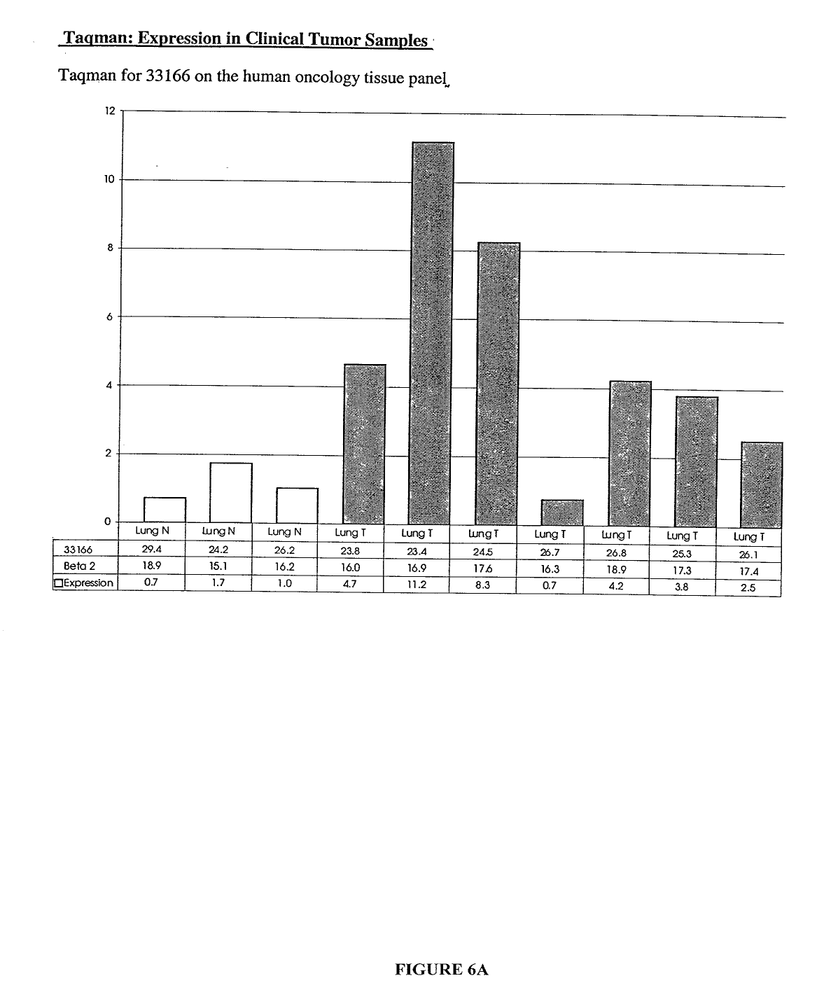

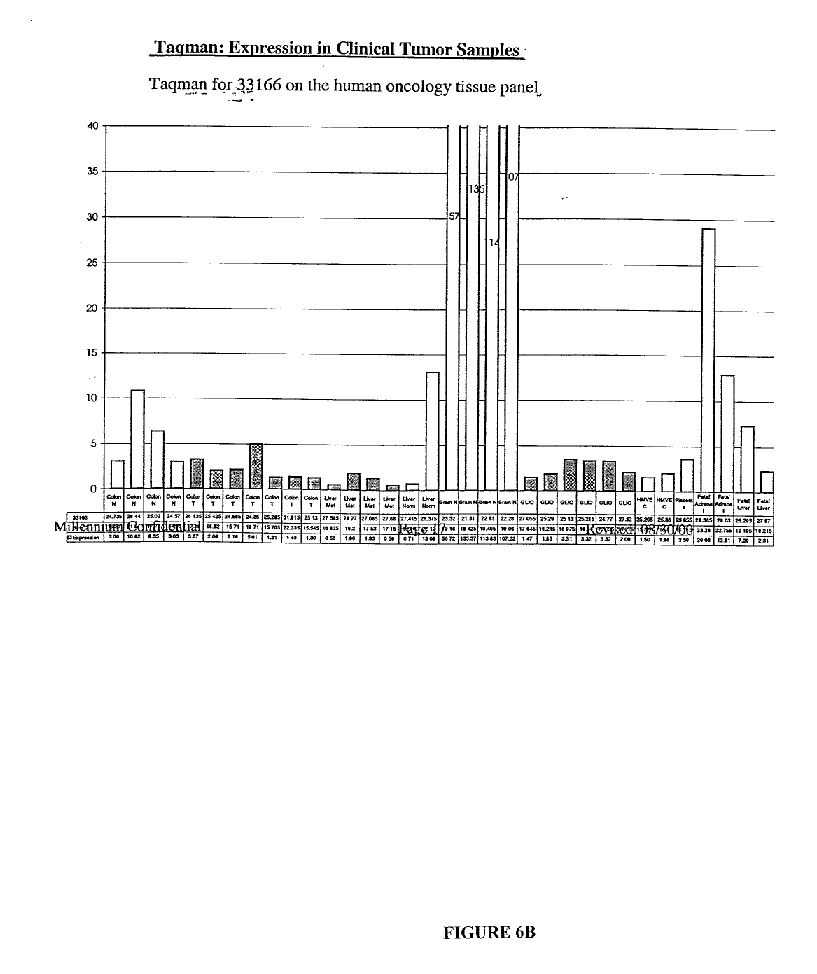

In particular, the 33166 gene is associated with lung and breast cancer. 33166 was identified as being expressed at high levels in human breast carcinoma samples in comparison to normal human breast tissue samples (FIGS. 5a and 5b). Also, as revealed by Taqman data, 33166 was modestly upregulated in some breast and lung tumors in comparison to normal breast and lung tissues (FIGS. 5a and 5b and 6). Inhibition of this alpha/beta hydrolase may inhibit tumor progression.

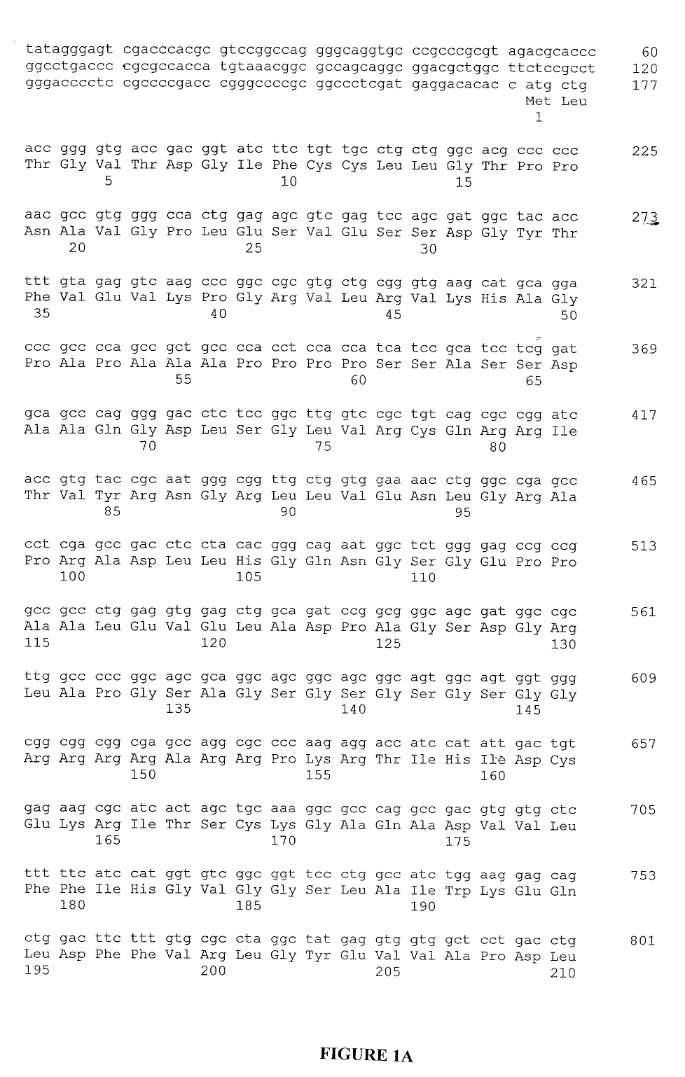

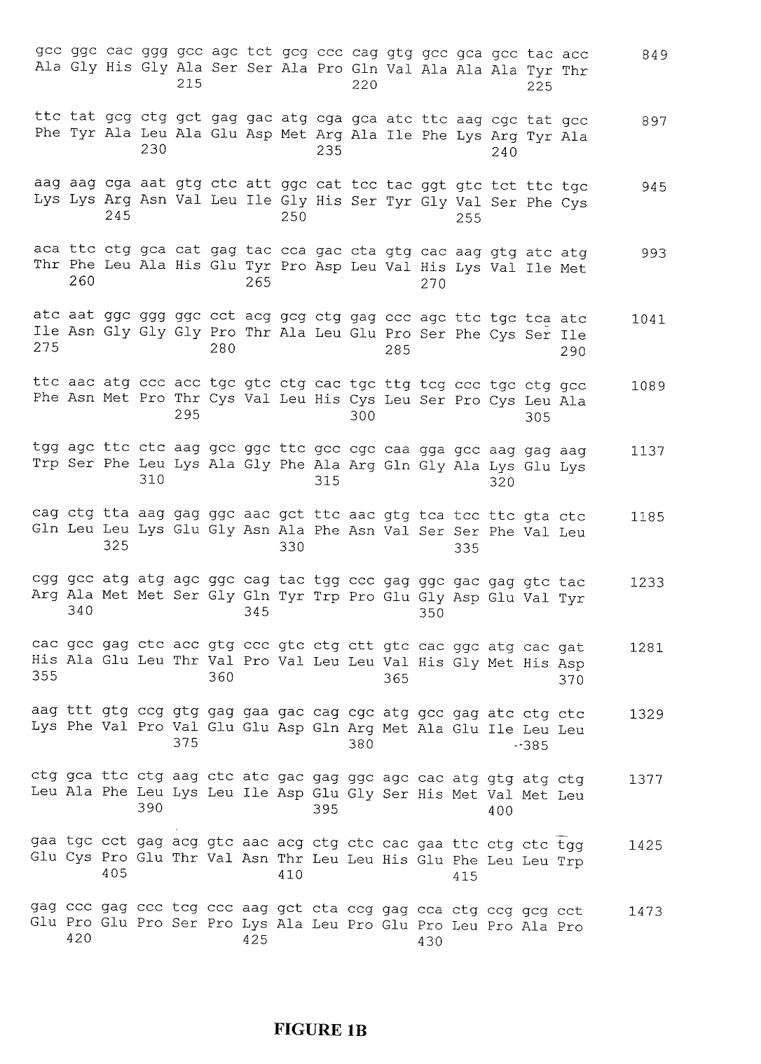

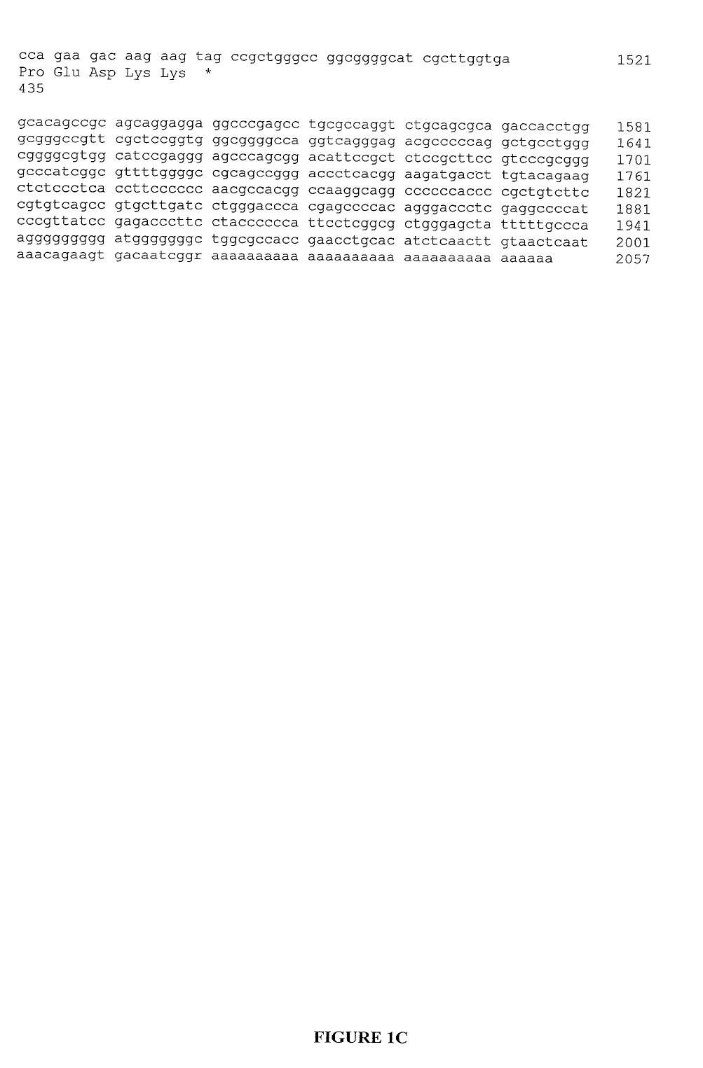

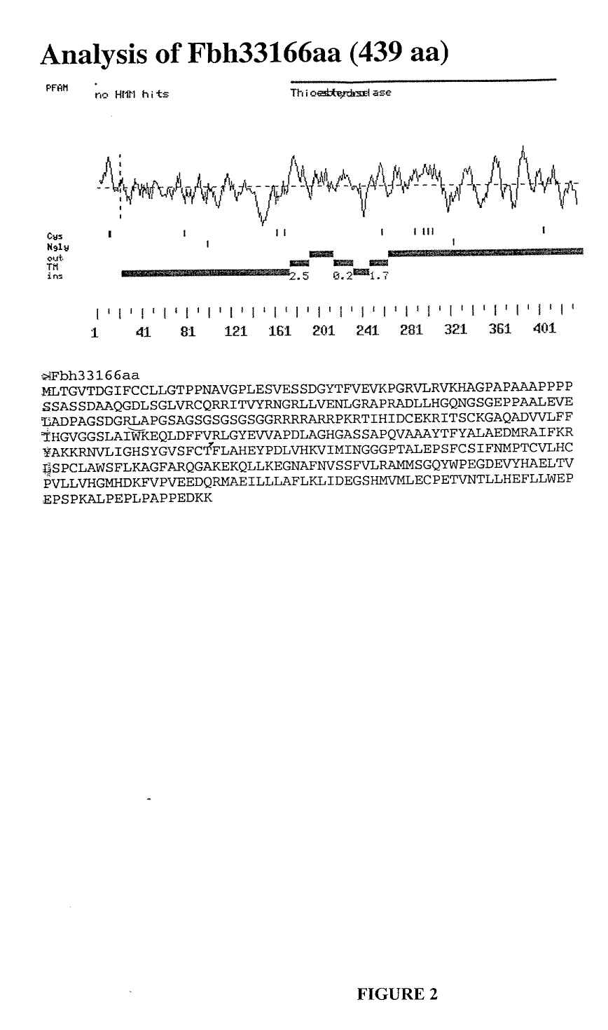

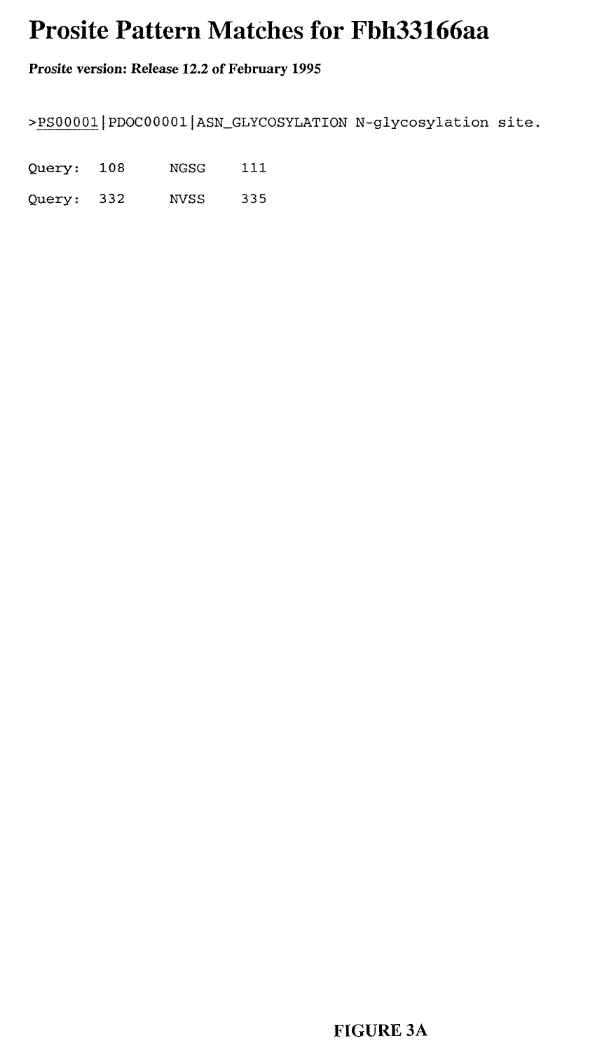

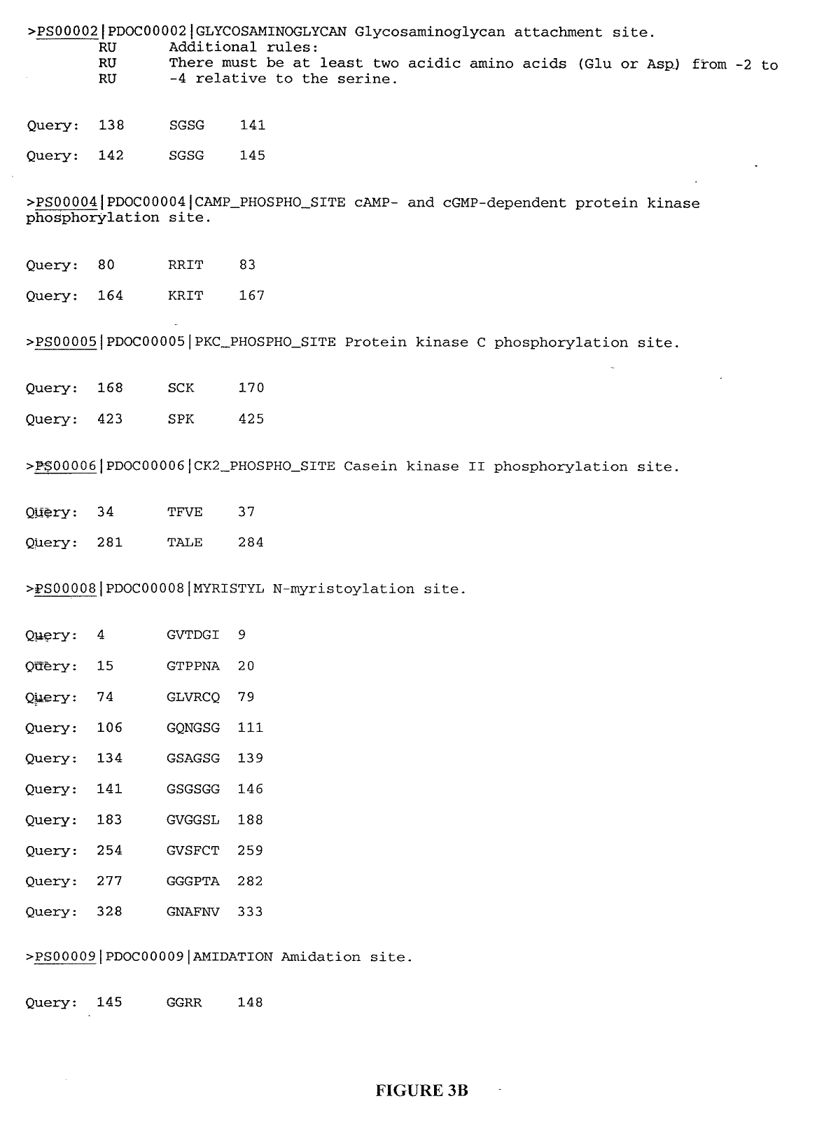

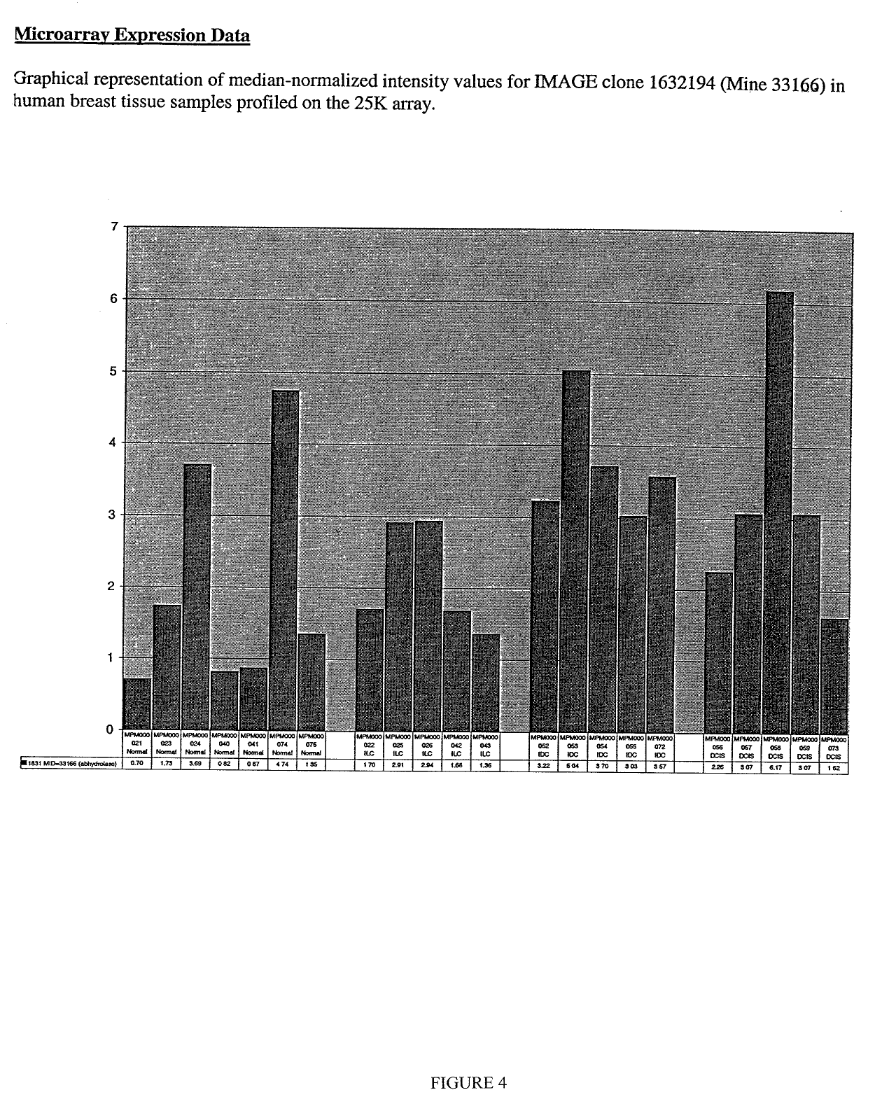

The alpha/beta hydrolase-like gene, clone 33166, was identified in a primary human ostaoblast cDNA library. Clone 33166 encodes an approximately 2.1 Kb mRNA transcript having the corresponding cDNA set forth in FIG. 1 (SEQ ID NO:1). This transcript has a 1320 nucleotide open reading frame (nucleotides 176-1495 of SEQ ID NO:1 corresponding to nucleotides designated 1-1320 in FIG. 1), which encodes a 439 amino acid protein (FIG. 1, SEQ ID NO:2) having a molecular weight of approximately 48.2 kDa. An analysis of the full-length 33166 polypeptide predicts that the N-terminal 21 amino acids represent a signal peptide. Transmembrane segments from amino acids (aa) 174-191, 214-231, and 247-263 were predicted by MEMSAT. Transmembrane segments were also predicted from aa 154-171, 194-211, and 227-243 of the presumed mature peptide sequence. Prosite program analysis was used to predict various sites within the 33166 protein. N-glycosylation sites were predicted at aa 108-111, and 332-335. Glycosaminoglycan attachment sites were predicted at aa 138-141, and aa 142-145. cAMP- and cGMP-dependent protein kinase phosphorylation sites were predicted at aa 80-83 and 164-167. Protein kinase C phosphorylation sites were predicted at aa 168-170, and 423-425. Casein kinase II phosphorylation sites were predicted at aa 34-37, and 281-284. N-myristoylation sites were predicted at aa 4-9, 15-20, 74-79, 106-111, 134-139, 141-146, 183-188, 254-259, 277-282, and 328-333. An amidation site was predicted at aa 145-148.

The alpha/beta hydrolase-like protein possesses an alpha/beta hydrolase domain, from aa 203-416, as predicted by HMMer, Version 2. The canonical form of this domain has a core topology of eight beta-sheets connected by alpha-helices, and a conserved catalytic triad (Ollis et al. (1992) Protein Eng 5(3): 197-211). This topology has been expanded to encompasses other variations; however, the catalytic triad of nucleophilic-, acidic-, and histidine residues are conserved as described herein. See for example, Heikinheimo et al. (1999) Structure 7(6): R141-R146; the ESTHER database (http://meleze.ensam.inra.fr/cholinesterase/).

The alpha/beta hydrolase-like protein displays identity to several ProDom consensus sequences including 29% identity to a carboxylesterase sequence over a 131 amino acid overlap ; 27% identity to an epoxide hydrolase sequence over a 90 amino acid overlap; 22% identity to a lipase sequence over a 131 amino acid overlap; 30% identity over a 99 amino acid overlap; 26% identity over a 129 amino acid overlap; and 25% identity to a DNA polymerase over a 112 amino acid overlap. Examples of proteins comprising domains from each of these consensus sequences include hypothetical proteins of Escherichia coli; E1-E2 ATPases of Mycobacterium tuberculosis and Sacchromyces cerevisiae; a putative esterase/lipase from Mycoplasma genitalium; a hypothetical protein from Methanococcus jannaschi; a protein kinase-like protein from Arabidopsis thaliana; and a Mycobacteriophage TM4 protein respectively.

A plasmid containing the 33166 cDNA insert was deposited with American Type Culture Collection (ATCC), 10801 University Boulevard, Manassas, Va., on ______, and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. [symbol] 112.

The alpha/beta hydrolase-like sequences of the invention are members of a family of molecules having conserved structural features. The term “family” when referring to the proteins and nucleic acid molecules of the invention is intended to mean two or more proteins or nucleic acid molecules having sufficient amino acid or nucleotide sequence identity as defined herein. Such family members can be naturally occurring and can be from either the same or different species. For example, a family can contain a first protein of murine origin and a homologue of that protein of human origin, as well as a second, distinct protein of human origin and a murine homologue of that protein. Members of a family may also have common functional characteristics.

Preferred alpha/beta hydrolase-like polypeptides of the present invention have an amino acid sequence sufficiently identical to the amino acid sequence of FIG. 1 (SEQ ID NO:2). The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 95%, or 98% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to alpha/beta hydrolase-like nucleic acid molecules of the invention. BLAST protein searches can be performed with the XBLAST program, score=50, wordlength=3, to obtain amino acid sequences homologous to alpha/beta hydrolase-like protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Accordingly, another embodiment of the invention features isolated alpha/beta hydrolase-like proteins and polypeptides having an alpha/beta hydrolase-like protein activity. As used interchangeably herein, a “alpha/beta hydrolase-like protein activity”, “biological activity of an alpha/beta hydrolase-like protein”, or “functional activity of an alpha/beta hydrolase-like protein” refers to an activity exerted by an alpha/beta hydrolase-like protein, polypeptide, or nucleic acid molecule on an alpha/beta hydrolase-like responsive cell as determined in vivo, or in vitro, according to standard assay techniques. An alpha/beta hydrolase-like activity can be a direct activity, such as an association with or an enzymatic activity on a second protein, or an indirect activity, such as a cellular signaling activity mediated by interaction of the alpha/beta hydrolase-like protein with a second protein. In a preferred embodiment, an alpha/beta hydrolase-like activity includes at least one or more of the following activities: (1) modulating (stimulating and/or enhancing or inhibiting) cellular cycle regulation, proliferation, differentiation, growth and/or function (2) modulating lipid and cholesterol metabolism; (3) modulating biotransformation of drugs and other chemicals; 4) modulating detoxification, particularly of aromatic compounds; 5) modulating neurotransmission; 6) modulating an enzyme activity selected from a lipase, esterase, and/or a protease activity.

An “isolated” or “purified” alpha/beta hydrolase-like nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5N and 3N ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated alpha/beta hydrolase-like nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. An alpha/beta hydrolase-like protein that is substantially free of cellular material includes preparations of alpha/beta hydrolase-like protein having less than about 30%, 20%, 10%, or 5% (by dry weight) of non-alpha/beta hydrolase-like protein (also referred to herein as a “contaminating protein”). When the alpha/beta hydrolase-like protein or biologically active portion thereof is recombinantly produced, preferably, culture medium represents less than about 30%, 20%, 10%, or 5% of the volume of the protein preparation. When alpha/beta hydrolase-like protein is produced by chemical synthesis, preferably the protein preparations have less than about 30%, 20%, 10%, or 5% (by dry weight) of chemical precursors or non-alpha/beta hydrolase-like chemicals.

Various aspects of the invention are described in further detail in the following subsections.

I. Isolated Nucleic Acid Molecules

One aspect of the invention pertains to isolated nucleic acid molecules comprising nucleotide sequences encoding alpha/beta hydrolase-like proteins and polypeptides or biologically active portions thereof, as well as nucleic acid molecules sufficient for use as hybridization probes to identify alpha/beta hydrolase-like-encoding nucleic acids (e.g., alpha/beta hydrolase-like mRNA) and fragments for use as PCR primers for the amplification or mutation of alpha/beta hydrolase-like nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequences encoding the alpha/beta hydrolase-like proteins of the present invention include sequences set forth in SEQ ID NO:1, the nucleotide sequence of the cDNA insert of the plasmid deposited with the ATCC as Accession Number ______ (the “cDNA of ATCC ______”), and complements thereof. By “complement” is intended a nucleotide sequence that is sufficiently complementary to a given nucleotide sequence such that it can hybridize to the given nucleotide sequence to thereby form a stable duplex. The corresponding amino acid sequence for the alpha/beta hydrolase-like protein encoded by these nucleotide sequences is set forth in SEQ ID NO:2. The invention also encompasses nucleic acid molecules comprising nucleotide sequences encoding partial-length alpha/beta hydrolase-like proteins, including the sequence set forth in SEQ ID NO:1, and complements thereof.

Nucleic acid molecules that are fragments of these alpha/beta hydrolase-like nucleotide sequences are also encompassed by the present invention. By “fragment” is intended a portion of the nucleotide sequence encoding an alpha/beta hydrolase-like protein. A fragment of an alpha/beta hydrolase-like nucleotide sequence may encode a biologically active portion of an alpha/beta hydrolase-like protein, or it may be a fragment that can be used as a hybridization probe or PCR primer using methods disclosed below. A biologically active portion of an alpha/beta hydrolase-like protein can be prepared by isolating a portion of one of the 33166 nucleotide sequences of the invention, expressing the encoded portion of the alpha/beta hydrolase-like protein (e.g., by recombinant expression in vitro), and assessing the activity of the encoded portion of the alpha/beta hydrolase-like protein. Nucleic acid molecules that are fragments of an alpha/beta hydrolase-like nucleotide sequence comprise at least about 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400nucleotides, or up to the number of nucleotides present in a full-length alpha/beta hydrolase-like nucleotide sequence disclosed herein (for example, 1851 nucleotides for SEQ ID NO:1, respectively) depending upon the intended use.

It is understood that isolated fragments include any contiguous sequence not disclosed prior to the invention as well as sequences that are substantially the same and which are not disclosed. Accordingly, if an isolated fragment is disclosed prior to the present invention, that fragment is not intended to be encompassed by the invention. When a sequence is not disclosed prior to the present invention, an isolated nucleic acid fragment is at least about 12, 15, 20, 25, or 30 contiguous nucleotides. Other regions of the nucleotide sequence may comprise fragments of various sizes, depending upon potential homology with previously disclosed sequences.

A fragment of an alpha/beta hydrolase-like nucleotide sequence that encodes a biologically active portion of an alpha/beta hydrolase-like protein of the invention will encode at least about 15, 25, 30, 50, 75, 100, 125, 150, 175, 200, 250, or 300 contiguous amino acids, or up to the total number of amino acids present in a full-length alpha/beta hydrolase-like protein of the invention (for example, 439 amino acids for SEQ ID NO:2. Fragments of an alpha/beta hydrolase-like nucleotide sequence that are useful as hybridization probes for PCR primers generally need not encode a biologically active portion of an alpha/beta hydrolase-like protein.

Nucleic acid molecules that are variants of the alpha/beta hydrolase-like nucleotide sequences disclosed herein are also encompassed by the present invention. “Variants” of the alpha/beta hydrolase-like nucleotide sequences include those sequences that encode the alpha/beta hydrolase-like proteins disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the alpha/beta hydrolase-like proteins disclosed in the present invention as discussed below. Generally, nucleotide sequence variants of the invention will have at least about 45%, 55%, 65%, 75%, 85%, 95%, or 98% identity to a particular nucleotide sequence disclosed herein. A variant alpha/beta hydrolase-like nucleotide sequence will encode an alpha/beta hydrolase-like protein that has an amino acid sequence having at least about 45%, 55%, 65%, 75%, 85%, 95%, or 98% identity to the amino acid sequence of an alpha/beta hydrolase-like protein disclosed herein.

In addition to the alpha/beta hydrolase-like nucleotide sequences shown in SEQ ID NOs: 1 and 3, and the nucleotide sequence of the cDNA of ATCC ______, it will be appreciated by those skilled in the art that DNA sequence polymorphisms that lead to changes in the amino acid sequences of alpha/beta hydrolase-like proteins may exist within a population (e.g., the human population). Such genetic polymorphism in an alpha/beta hydrolase-like gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding an alpha/beta hydrolase-like protein, preferably a mammalian alpha/beta hydrolase-like protein. As used herein, the phrase “allelic variant” refers to a nucleotide sequence that occurs at an alpha/beta hydrolase-like locus or to a polypeptide encoded by the nucleotide sequence. Such natural allelic variations can typically result in 1-5% variance in the nucleotide sequence of the alpha/beta hydrolase-like gene. Any and all such nucleotide variations and resulting amino acid polymorphisms or variations in an alpha/beta hydrolase-like sequence that are the result of natural allelic variation and that do not alter the functional activity of alpha/beta hydrolase-like proteins are intended to be within the scope of the invention.

Moreover, nucleic acid molecules encoding alpha/beta hydrolase-like proteins from other species (alpha/beta hydrolase-like homologues), which have a nucleotide sequence differing from that of the alpha/beta hydrolase-like sequences disclosed herein, are intended to be within the scope of the invention. For example, nucleic acid molecules corresponding to natural allelic variants and homologues of the human alpha/beta hydrolase-like cDNA of the invention can be isolated based on their identity to the human alpha/beta hydrolase-like nucleic acid disclosed herein using the human cDNA, or a portion thereof, as a hybridization probe according to standard hybridization techniques under stringent hybridization conditions as disclosed below.

In addition to naturally-occurring allelic variants of the alpha/beta hydrolase-like sequences that may exist in the population, the skilled artisan will further appreciate that changes can be introduced by mutation into the nucleotide sequences of the invention thereby leading to changes in the amino acid sequence of the encoded alpha/beta hydrolase-like proteins, without altering the biological activity of the alpha/beta hydrolase-like proteins. Thus, an isolated nucleic acid molecule encoding an alpha/beta hydrolase-like protein having a sequence that differs from that of SEQ ID NO:2 can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, preferably, conservative amino acid substitutions may be made at one or more predicted, preferably nonessential amino acid residues. A “nonessential” amino acid residue is a residue that can be altered from the wild-type sequence of an alpha/beta hydrolase-like protein (e.g., the sequence of SEQ ID NO:2) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif, where such residues are essential for protein activity.

Alternatively, variant alpha/beta hydrolase-like nucleotide sequences can be made by introducing mutations randomly along all or part of an alpha/beta hydrolase-like coding sequence, such as by saturation mutagenesis, and the resultant mutants can be screened for alpha/beta hydrolase-like biological activity to identify mutants that retain activity. Following mutagenesis, the encoded protein can be expressed recombinantly, and the activity of the protein can be determined using standard assay techniques.

Thus the nucleotide sequences of the invention include the sequences disclosed herein as well as fragments and variants thereof. The alpha/beta hydrolase-like nucleotide sequences of the invention, and fragments and variants thereof, can be used as probes and/or primers to identify and/or clone alpha/beta hydrolase-like homologues in other cell types, e.g., from other tissues, as well as alpha/beta hydrolase-like homologues from other mammals. Such probes can be used to detect transcripts or genomic sequences encoding the same or identical proteins. These probes can be used as part of a diagnostic test kit for identifying cells or tissues that misexpress an alpha/beta hydrolase-like protein, such as by measuring levels of an alpha/beta hydrolase-like-encoding nucleic acid in a sample of cells from a subject, e.g., detecting alpha/beta hydrolase-like mRNA levels or determining whether a genomic alpha/beta hydrolase-like gene has been mutated or deleted.

In this manner, methods such as PCR, hybridization, and the like can be used to identify such sequences having substantial identity to the sequences of the invention. See, for example, Sambrook et al. (1989) Molecular Cloning: Laboratory Manual (2d ed., Cold Spring Harbor Laboratory Press, Plainview, N.Y.) and Innis, et al. (1990) PCR Protocols: A Guide to Methods and Applications (Academic Press, N.Y.). alpha/beta hydrolase-like nucleotide sequences isolated based on their sequence identity to the alpha/beta hydrolase-like nucleotide sequences set forth herein or to fragments and variants thereof are encompassed by the present invention.

In a hybridization method, all or part of a known alpha/beta hydrolase-like nucleotide sequence can be used to screen cDNA or genomic libraries. Methods for construction of such cDNA and genomic libraries are generally known in the art and are disclosed in Sambrook et al. (1989) Molecular Cloning: A Laboratory Manual (2d ed., Cold Spring Harbor Laboratory Press, Plainview, N.Y.). The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known alpha/beta hydrolase-like nucleotide sequence disclosed herein. Degenerate primers designed on the basis of conserved nucleotides or amino acid residues in a known alpha/beta hydrolase-like nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, or 400 consecutive nucleotides of an alpha/beta hydrolase-like nucleotide sequence of the invention or a fragment or variant thereof. Preparation of probes for hybridization is generally known in the art and is disclosed in Sambrook et al. (1989) Molecular Cloning: A Laboratory Manual (2d ed., Cold Spring Harbor Laboratory Press, Plainview, N.Y.), herein incorporated by reference.

For example, in one embodiment, a previously unidentified alpha/beta hydrolase-like nucleic acid molecule hybridizes under stringent conditions to a probe that is a nucleic acid molecule comprising one of the alpha/beta hydrolase-like nucleotide sequences of the invention or a fragment thereof. In another embodiment, the previously unknown alpha/beta hydrolase-like nucleic acid molecule is at least about 300, 325, 350, 375, 400, 425, 450, 500, 550, 600, 650, 700, 800, 900, 1000, 2,000, 3,000, 4,000 or 5,000 nucleotides in length and hybridizes under stringent conditions to a probe that is a nucleic acid molecule comprising one of the alpha/beta hydrolase-like nucleotide sequences disclosed herein or a fragment thereof.

Accordingly, in another embodiment, an isolated previously unknown alpha/beta hydrolase-like nucleic acid molecule of the invention is at least about 300, 325, 350, 375, 400, 425, 450, 500, 550, 600, 650, 700, 800, 900, 1000, 1,100, 1,200, 1,300, or 1,400 nucleotides in length and hybridizes under stringent conditions to a probe that is a nucleic acid molecule comprising one of the nucleotide sequences of the invention, preferably the coding sequence set forth in SEQ ID NO:1, the cDNA of ATCC ______, or a complement, fragment, or variant thereof.

As used herein, the term “hybridizes under stringent conditions” is intended to describe conditions for hybridization and washing under which nucleotide sequences having at least about 60%, 65%, 70%, preferably 75% identity to each other typically remain hybridized to each other. Such stringent conditions are known to those skilled in the art and can be found in Current Protocols in Molecular Biology (John Wiley & Sons, New York (1989)), 6.3.1-6.3.6. A preferred, non-limiting example of stringent hybridization conditions is hybridization in 6× sodium chloride/sodium citrate (SSC) at about 45EC, followed by one or more washes in 0.2× SSC, 0.1% SDS at 50-65EC. In another preferred embodiment, stringent conditions comprise hybridization in 6× SSC at 42EC, followed by washing with 1× SSC at 55EC. Preferably, an isolated nucleic acid molecule that hybridizes under stringent conditions to an alpha/beta hydrolase-like sequence of the invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Thus, in addition to the alpha/beta hydrolase-like nucleotide sequences disclosed herein and fragments and variants thereof, the isolated nucleic acid molecules of the invention also encompass homologous DNA sequences identified and isolated from other cells and/or organisms by hybridization with entire or partial sequences obtained from the alpha/beta hydrolase-like nucleotide sequences disclosed herein or variants and fragments thereof.

The present invention also encompasses antisense nucleic acid molecules, i.e., molecules that are complementary to a sense nucleic acid encoding a protein, e.g., complementary to the coding strand of a double-stranded cDNA molecule, or complementary to an mRNA sequence. Accordingly, an antisense nucleic acid can hydrogen bond to a sense nucleic acid. The antisense nucleic acid can be complementary to an entire alpha/beta hydrolase-like coding strand, or to only a portion thereof, e.g., all or part of the protein coding region (or open reading frame). An antisense nucleic acid molecule can be antisense to a noncoding region of the coding strand of a nucleotide sequence encoding an alpha/beta hydrolase-like protein. The noncoding regions are the 5N and 3N sequences that flank the coding region and are not translated into amino acids.

Given the coding-strand sequence encoding an alpha/beta hydrolase-like protein disclosed herein (e.g., SEQ ID NO:1), antisense nucleic acids of the invention can be designed according to the rules of Watson and Crick base pairing. The antisense nucleic acid molecule can be complementary to the entire coding region of alpha/beta hydrolase-like mRNA, but more preferably is an oligonucleotide that is antisense to only a portion of the coding or noncoding region of alpha/beta hydrolase-like mRNA. For example, the antisense oligonucleotide can be complementary to the region surrounding the translation start site of alpha/beta hydrolase-like mRNA. An antisense oligonucleotide can be, for example, about 5, 10, 15, 20, 25, 30, 35, 40, 45, or 50 nucleotides in length. An antisense nucleic acid of the invention can be constructed using chemical synthesis and enzymatic ligation procedures known in the art.

For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, including, but not limited to, for example e.g., phosphorothioate derivatives and acridine substituted nucleotides. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When used therapeutically, the antisense nucleic acid molecules of the invention are typically administered to a subject or generated in situ such that they hybridize with or bind to cellular mRNA and/or genomic DNA encoding an alpha/beta hydrolase-like protein to thereby inhibit expression of the protein, e.g., by inhibiting transcription and/or translation. An example of a route of administration of antisense nucleic acid molecules of the invention includes direct injection at a tissue site. Alternatively, antisense nucleic acid molecules can be modified to target selected cells and then administered systemically. For example, antisense molecules can be linked to peptides or antibodies to form a complex that specifically binds to receptors or antigens expressed on a selected cell surface. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred. These antisense constructs can be useful in the treatment of lung and breast cancer.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes, which are catalytic RNA molecules with ribonuclease activity that are capable of cleaving a single-stranded nucleic acid, such as an mRNA, to which they have a complementary region. Ribozymes (e.g., hammerhead ribozymes (described in Haselhoff and Gerlach (1988) Nature 334:585-591)) can be used to catalytically cleave alpha/beta hydrolase-like mRNA transcripts to thereby inhibit translation of alpha/beta hydrolase-like mRNA. A ribozyme having specificity for an alpha/beta hydrolase-like-encoding nucleic acid can be designed based upon the nucleotide sequence of an alpha/beta hydrolase-like cDNA disclosed herein (e.g., SEQ ID NO:1). See, e.g., Cech et al., U.S. Pat. No. 4,987,071; and Cech et al., U.S. Pat. No. 5,116,742. Alternatively, alpha/beta hydrolase-like mRNA can be used to select a catalytic RNA having a specific ribonuclease activity from a pool of RNA molecules. See, e.g., Bartel and Szostak (1993) Science 261:1411-1418.

The invention also encompasses nucleic acid molecules that form triple helical structures. For example, alpha/beta hydrolase-like gene expression can be inhibited by targeting nucleotide sequences complementary to the regulatory region of the alpha/beta hydrolase-like protein (e.g., the alpha/beta hydrolase-like promoter and/or enhancers) to form triple helical structures that prevent transcription of the alpha/beta hydrolase-like gene in target cells. See generally Helene (1991) Anticancer Drug Des. 6(6): 569; Helene (1992) Ann. N.Y. Acad. Sci. 660:27; and Maher (1992) Bioassays 14(12): 807.

In preferred embodiments, the nucleic acid molecules of the invention can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid-phase peptide synthesis protocols as described, for example, in Hyrup et al. (1996), supra; Perry-O'Keefe et al. (1996) Proc. Natl. Acad. Sci. USA 93:14670.

PNAs of an alpha/beta hydrolase-like molecule can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, e.g., in the analysis of single base pair mutations in a gene by, e.g., PNA-directed PCR clamping; as artificial restriction enzymes when used in combination with other enzymes, e.g., S1 nucleases (Hyrup (1996), supra); or as probes or primers for DNA sequence and hybridization (Hyrup (1996), supra; Perry-O'Keefe et al. (1996), supra).

In another embodiment, PNAs of an alpha/beta hydrolase-like molecule can be modified, e.g., to enhance their stability, specificity, or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra; Finn et al. (1996) Nucleic Acids Res. 24(17): 3357-63; Mag et al. (1989) Nucleic Acids Res. 17:5973; and Peterson et al. (1975) Bioorganic Med. Chem. Lett. 5:1119.

II. Isolated Alpha/Beta Hydrolase-Like Proteins and Anti-Alpha/Beta Hydrolase-Like Antibodies

Alpha/beta hydrolase-like proteins are also encompassed within the present invention. By “alpha/beta hydrolase-like protein” is intended a protein having the amino acid sequence set forth in SEQ ID NO:2, as well as fragments, biologically active portions, and variants thereof.

“Fragments” or “biologically active portions” include polypeptide fragments suitable for use as immunogens to raise anti-alpha/beta hydrolase-like antibodies. Fragments include peptides comprising amino acid sequences sufficiently identical to or derived from the amino acid sequence of an alpha/beta hydrolase-like protein, or partial-length protein, of the invention and exhibiting at least one activity of an alpha/beta hydrolase-like protein, but which include fewer amino acids than the full-length (SEQ ID NO:2) or alpha/beta hydrolase-like protein disclosed herein. Typically, biologically active portions comprise a domain or motif with at least one activity of the alpha/beta hydrolase-like protein. A biologically active portion of an alpha/beta hydrolase-like protein can be a polypeptide which is, for example, 10, 25, 50, 100 or more amino acids in length. Such biologically active portions can be prepared by recombinant techniques and evaluated for one or more of the functional activities of a native alpha/beta hydrolase-like protein. As used here, a fragment comprises at least 5 contiguous amino acids of SEQ ID NO:2. The invention encompasses other fragments, however, such as any fragment in the protein greater than 6, 7, 8, or 9 amino acids.

By “variants” is intended proteins or polypeptides having an amino acid sequence that is at least about 45%, 55%, 65%, preferably about 75%, 85%, 95%, or 98% identical to the amino acid sequence of SEQ ID NO:2. Variants also include polypeptides encoded by the cDNA insert of the plasmid deposited with ATCC as Accession Number ______, or polypeptides encoded by a nucleic acid molecule that hybridizes to the nucleic acid molecule of SEQ ID NO:1, or a complement thereof, under stringent conditions. Such variants generally retain the functional activity of the alpha/beta hydrolase-like proteins of the invention. Variants include polypeptides that differ in amino acid sequence due to natural allelic variation or mutagenesis.

The invention also provides alpha/beta hydrolase-like chimeric or fusion proteins. As used herein, an alpha/beta hydrolase-like “chimeric protein” or “fusion protein” comprises an alpha/beta hydrolase-like polypeptide operably linked to a non-alpha/beta hydrolase-like polypeptide. A “alpha/beta hydrolase-like polypeptide” refers to a polypeptide having an amino acid sequence corresponding to an alpha/beta hydrolase-like protein, whereas a “non-alpha/beta hydrolase-like polypeptide” refers to a polypeptide having an amino acid sequence corresponding to a protein that is not substantially identical to the alpha/beta hydrolase-like protein, e.g., a protein that is different from the alpha/beta hydrolase-like protein and which is derived from the same or a different organism. Within an alpha/beta hydrolase-like fusion protein, the alpha/beta hydrolase-like polypeptide can correspond to all or a portion of an alpha/beta hydrolase-like protein, preferably at least one biologically active portion of an alpha/beta hydrolase-like protein. Within the fusion protein, the term “operably linked” is intended to indicate that the alpha/beta hydrolase-like polypeptide and the non-alpha/beta hydrolase-like polypeptide are fused in-frame to each other. The non-alpha/beta hydrolase-like polypeptide can be fused to the N-terminus or C-terminus of the alpha/beta hydrolase-like polypeptide.

One useful fusion protein is a GST-alpha/beta hydrolase-like fusion protein in which the alpha/beta hydrolase-like sequences are fused to the C-terminus of the GST sequences. Such fusion proteins can facilitate the purification of recombinant alpha/beta hydrolase-like proteins.

In yet another embodiment, the fusion protein is an alpha/beta hydrolase-like-immunoglobulin fusion protein in which all or part of an alpha/beta hydrolase-like protein is fused to sequences derived from a member of the immunoglobulin protein family. The alpha/beta hydrolase-like-immunoglobulin fusion proteins of the invention can be incorporated into pharmaceutical compositions and administered to a subject to inhibit an interaction between an alpha/beta hydrolase-like ligand and an alpha/beta hydrolase-like protein on the surface of a cell, thereby suppressing alpha/beta hydrolase-like-mediated signal transduction in vivo. The alpha/beta hydrolase-like-immunoglobulin fusion proteins can be used to affect the bioavailability of an alpha/beta hydrolase-like cognate ligand. Inhibition of the alpha/beta hydrolase-like ligand/alpha/beta hydrolase-like interaction may be useful therapeutically, both for treating proliferative and differentiative disorders and for modulating (e.g., promoting or inhibiting) cell survival. Moreover, the alpha/beta hydrolase-like-immunoglobulin fusion proteins of the invention can be used as immunogens to produce anti-alpha/beta hydrolase-like antibodies in a subject, to purify alpha/beta hydrolase-like ligands, and in screening assays to identify molecules that inhibit the interaction of an alpha/beta hydrolase-like protein with an alpha/beta hydrolase-like ligand.

Preferably, an alpha/beta hydrolase-like chimeric or fusion protein of the invention is produced by standard recombinant DNA techniques. For example, DNA fragments coding for the different polypeptide sequences may be ligated together in-frame, or the fusion gene can be synthesized, such as with automated DNA synthesizers. Alternatively, PCR amplification of gene fragments can be carried out using anchor primers that give rise to complementary overhangs between two consecutive gene fragments, which can subsequently be annealed and reamplified to generate a chimeric gene sequence (see, e.g., Ausubel et al., eds. (1995) Current Protocols in Molecular Biology) (Greene Publishing and Wiley-Interscience, NY). Moreover, an alpha/beta hydrolase-like-encoding nucleic acid can be cloned into a commercially available expression vector such that it is linked in-frame to an existing fusion moiety.

Variants of the alpha/beta hydrolase-like proteins can function as either alpha/beta hydrolase-like agonists (mimetics) or as alpha/beta hydrolase-like antagonists. Variants of the alpha/beta hydrolase-like protein can be generated by mutagenesis, e.g., discrete point mutation or truncation of the alpha/beta hydrolase-like protein. An agonist of the alpha/beta hydrolase-like protein can retain substantially the same, or a subset, of the biological activities of the naturally occurring form of the alpha/beta hydrolase-like protein. An antagonist of the alpha/beta hydrolase-like protein can inhibit one or more of the activities of the naturally occurring form of the alpha/beta hydrolase-like protein by, for example, competitively binding to a downstream or upstream member of a cellular signaling cascade that includes the alpha/beta hydrolase-like protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the alpha/beta hydrolase-like proteins.

Variants of an alpha/beta hydrolase-like protein that function as either alpha/beta hydrolase-like agonists or as alpha/beta hydrolase-like antagonists can be identified by screening combinatorial libraries of mutants, e.g., truncation mutants, of an alpha/beta hydrolase-like protein for alpha/beta hydrolase-like protein agonist or antagonist activity. In one embodiment, a variegated library of alpha/beta hydrolase-like variants is generated by combinatorial mutagenesis at the nucleic acid level and is encoded by a variegated gene library. A variegated library of alpha/beta hydrolase-like variants can be produced by, for example, enzymatically ligating a mixture of synthetic oligonucleotides into gene sequences such that a degenerate set of potential alpha/beta hydrolase-like sequences is expressible as individual polypeptides, or alternatively, as a set of larger fusion proteins (e.g., for phage display) containing the set of alpha/beta hydrolase-like sequences therein. There are a variety of methods that can be used to produce libraries of potential alpha/beta hydrolase-like variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential alpha/beta hydrolase-like sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of an alpha/beta hydrolase-like protein coding sequence can be used to generate a variegated population of alpha/beta hydrolase-like fragments for screening and subsequent selection of variants of an alpha/beta hydrolase-like protein. In one embodiment, a library of coding sequence fragments can be generated by treating a double-stranded PCR fragment of an alpha/beta hydrolase-like coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the alpha/beta hydrolase-like protein.

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of alpha/beta hydrolase-like proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify alpha/beta hydrolase-like variants (Arkin and Yourvan (1992) Proc. Nat. Acad. Sci. USA 89:7811-7815; Delgrave et al. (1993) Protein Engineering 6(3): 327-331).

An isolated alpha/beta hydrolase-like polypeptide of the invention can be used as an immunogen to generate antibodies that bind alpha/beta hydrolase-like proteins using standard techniques for polyclonal and monoclonal antibody preparation. The full-length alpha/beta hydrolase-like protein can be used or, alternatively, the invention provides antigenic peptide fragments of alpha/beta hydrolase-like proteins for use as immunogens. The antigenic peptide of an alpha/beta hydrolase-like protein comprises at least 8, preferably 10, 15, 20, or 30 amino acid residues of the amino acid sequence shown in SEQ ID NO:2 and encompasses an epitope of an alpha/beta hydrolase-like protein such that an antibody raised against the peptide forms a specific immune complex with the alpha/beta hydrolase-like protein. Preferred epitopes encompassed by the antigenic peptide are regions of a alpha/beta hydrolase-like protein that are located on the surface of the protein, e.g., hydrophilic regions.

Accordingly, another aspect of the invention pertains to anti-alpha/beta hydrolase-like polyclonal and monoclonal antibodies that bind an alpha/beta hydrolase-like protein. Polyclonal anti-alpha/beta hydrolase-like antibodies can be prepared by immunizing a suitable subject (e.g., rabbit, goat, mouse, or other mammal) with an alpha/beta hydrolase-like immunogen. The anti-alpha/beta hydrolase-like antibody titer in the immunized subject can be monitored over time by standard techniques, such as with an enzyme linked immunosorbent assay (ELISA) using immobilized alpha/beta hydrolase-like protein. At an appropriate time after immunization, e.g., when the anti-alpha/beta hydrolase-like antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Alternative to preparing monoclonal antibody-secreting hybridomas, a monoclonal anti-alpha/beta hydrolase-like antibody can be identified and isolated by screening a recombinant combinatorial immunoglobulin library (e.g., an antibody phage display library) with an alpha/beta hydrolase-like protein to thereby isolate immunoglobulin library members that bind the alpha/beta hydrolase-like protein. Kits for generating and screening phage display libraries are commercially available (e.g., the Pharmacia Recombinant Phage Antibody System, Catalog No. 27-9400-01; and the Stratagene SurfZAP θ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-alpha/beta hydrolase-like antibodies, such as chimeric and humanized monoclonal antibodies, comprising both human and nonhuman portions, which can be made using standard recombinant DNA techniques, are within the scope of the invention. Such chimeric and humanized monoclonal antibodies can be produced by recombinant DNA techniques known in the art, for example using methods described in PCT Publication Nos. WO 86/101533 and WO 87/02671; European Patent Application Nos. 184,187, 171,496, 125,023, and 173,494; U.S. Pat. Nos. 4,816,567 and 5,225,539; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559); Morrson (1985) Science 229:1202-1207; Oi et al. (1986) Bio/Techniques 4:214; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

An anti-like antibody (e.g., monoclonal antibody) can be used to isolate alpha/beta hydrolase-like proteins by standard techniques, such as affinity chromatography or immunoprecipitation. An anti-alpha/beta hydrolase-like antibody can facilitate the purification of natural alpha/beta hydrolase-like protein from cells and of recombinantly produced alpha/beta hydrolase-like protein expressed in host cells. Moreover, an anti-alpha/beta hydrolase-like antibody can be used to detect alpha/beta hydrolase-like protein (e.g., in a cellular lysate or cell supernatant) in order to evaluate the abundance and pattern of expression of the alpha/beta hydrolase-like protein. Anti-alpha/beta hydrolase-like antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

III. Recombinant Expression Vectors and Host Cells

Another aspect of the invention pertains to vectors, preferably expression vectors, containing a nucleic acid encoding an alpha/beta hydrolase-like protein (or a portion thereof). “Vector” refers to a nucleic acid molecule capable of transporting another nucleic acid to which it has been linked, such as a “plasmid”, a circular double-stranded DNA loop into which additional DNA segments can be ligated, or a viral vector, where additional DNA segments can be ligated into the viral genome. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses), that serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, operably linked to the nucleic acid sequence to be expressed. “Operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., alpha/beta hydrolase-like proteins, mutant forms of alpha/beta hydrolase-like proteins, fusion proteins, etc.).

The recombinant expression vectors of the invention can be designed for expression of alpha/beta hydrolase-like protein in prokaryotic or eukaryotic host cells. Expression of proteins in prokaryotes is most often carried out in E. coli with vectors containing constitutive or inducible promoters directing the expression of either fusion or nonfusion proteins. Fusion vectors add a number of amino acids to a protein encoded therein, usually to the amino terminus of the recombinant protein. Typical fusion expression vectors include pGEX (Pharmacia Biotech Inc; Smith and Johnson (1988) Gene 67:31-40), pMAL (New England Biolabs, Beverly, Mass.), and pRIT5 (Pharmacia, Piscataway, N.J.) which fuse glutathione S-transferase (GST), maltose E binding protein, or protein A, respectively, to the target recombinant protein. Examples of suitable inducible nonfusion E. coli expression vectors include pTrc (Amann et al. (1988) Gene 69:301-315) and pET 11d (Studier et al. (1990) in Gene Expression Technology: Methods in Enzymology 185 (Academic Press, San Diego, Calif.), pp. 60-89). Strategies to maximize recombinant protein expression in E. coli can be found in Gottesman (1990) in Gene Expression Technology: Methods in Enzymology 185 (Academic Press, CA), pp. 119-128 and Wada et al. (1992) Nucleic Acids Res. 20:2111-2118. Target gene expression from the pTrc vector relies on host RNA polymerase transcription from a hybrid trp-lac fusion promoter.

Suitable eukaryotic host cells include insect cells (examples of Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cereivisiae include pYepSec1 (Baldari et al. (1987) EMBO J. 6:229-234), pMFa (Kuijan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook et al. (1989) Molecular cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.). Alternatively, the recombinant expression vector can be transcribed and translated in vitro, for example using T7 promoter regulatory sequences and T7 polymerase.

The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell but are still included within the scope of the term as used herein.

In one embodiment, the expression vector is a recombinant mammalian expression vector that comprises tissue-specific regulatory elements that direct expression of the nucleic acid preferentially in a particular cell type. Suitable tissue-specific promoters include the albumin promoter (e.g., liver-specific promoter; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Patent Publication No. 264,166). Developmentally-regulated promoters are also encompassed, for example the murine hox homeobox promoters (Kessel and Gruss (1990) Science 249:374-379), the α-fetoprotein promoter (Campes and Tilghman (1989) Genes Dev. 3:537-546), and the like.

The invention further provides a recombinant expression vector comprising a DNA molecule of the invention cloned into the expression vector in an antisense orientation. That is, the DNA molecule is operably linked to a regulatory sequence in a manner that allows for expression (by transcription of the DNA molecule) of an RNA molecule that is antisense to alpha/beta hydrolase-like mRNA. Regulatory sequences operably linked to a nucleic acid cloned in the antisense orientation can be chosen to direct the continuous expression of the antisense RNA molecule in a variety of cell types, for instance viral promoters and/or enhancers, or regulatory sequences can be chosen to direct constitutive, tissue-specific, or cell-type-specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (1986) Reviews—Trends in Genetics, Vol. 1(1).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (1989) Molecular Cloning: A Laboraty Manual (2d ed., Cold Spring Harbor Laboratory Press, Plainview, N.Y.) and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin, and methotrexate. Nucleic acid encoding a selectable marker can be introduced into a host cell on the same vector as that encoding an alpha/beta hydrolase-like protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) alpha/beta hydrolase-like protein. Accordingly, the invention further provides methods for producing alpha/beta hydrolase-like protein using the host cells of the invention. In one embodiment, the method comprises culturing the host cell of the invention, into which a recombinant expression vector encoding an alpha/beta hydrolase-like protein has been introduced, in a suitable medium such that alpha/beta hydrolase-like protein is produced. In another embodiment, the method further comprises isolating alpha/beta hydrolase-like protein from the medium or the host cell.

The host cells of the invention can also be used to produce nonhuman transgenic animals. For example, in one embodiment, a host cell of the invention is a fertilized oocyte or an embryonic stem cell into which alpha/beta hydrolase-like-coding sequences have been introduced. Such host cells can then be used to create nonhuman transgenic animals in which exogenous alpha/beta hydrolase-like sequences have been introduced into their genome or homologous recombinant animals in which endogenous alpha/beta hydrolase-like sequences have been altered. Such animals are useful for studying the function and/or activity of alpha/beta hydrolase-like genes and proteins and for identifying and/or evaluating modulators of alpha/beta hydrolase-like activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA that is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous alpha/beta hydrolase-like gene has been altered by homologous recombination between the endogenous gene and an exogenous DNA molecule introduced into a cell of the animal, e.g., an embryonic cell of the animal, prior to development of the animal.

A transgenic animal of the invention can be created by introducing alpha/beta hydrolase-like-encoding nucleic acid into the male pronuclei of a fertilized oocyte, e.g., by microinjection, retroviral infection, and allowing the oocyte to develop in a pseudopregnant female foster animal. The alpha/beta hydrolase-like cDNA sequence can be introduced as a transgene into the genome of a nonhuman animal. Alternatively, a homologue of the mouse alpha/beta hydrolase-like gene can be isolated based on hybridization and used as a transgene. Intronic sequences and polyadenylation signals can also be included in the transgene to increase the efficiency of expression of the transgene. A tissue-specific regulatory sequence(s) can be operably linked to the alpha/beta hydrolase-like transgene to direct expression of alpha/beta hydrolase-like protein to particular cells. Methods for generating transgenic animals via embryo manipulation and microinjection, particularly animals such as mice, have become conventional in the art and are described, for example, in U.S. Pat. Nos. 4,736,866, 4,870,009, and 4,873,191 and in Hogan (1986) Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the alpha/beta hydrolase-like transgene in its genome and/or expression of alpha/beta hydrolase-like mRNA in tissues or cells of the animals. A transgenic founder animal can then be used to breed additional animals carrying the transgene. Moreover, transgenic animals carrying a transgene encoding alpha/beta hydrolase-like gene can further be bred to other transgenic animals carrying other transgenes.

To create a homologous recombinant animal, one prepares a vector containing at least a portion of an alpha/beta hydrolase-like gene or a homolog of the gene into which a deletion, addition, or substitution has been introduced to thereby alter, e.g., functionally disrupt, the alpha/beta hydrolase-like gene. In a preferred embodiment, the vector is designed such that, upon homologous recombination, the endogenous alpha/beta hydrolase-like gene is functionally disrupted (i.e., no longer encodes a functional protein; also referred to as a “knock out” vector). Alternatively, the vector can be designed such that, upon homologous recombination, the endogenous alpha/beta hydrolase-like gene is mutated or otherwise altered but still encodes functional protein (e.g., the upstream regulatory region can be altered to thereby alter the expression of the endogenous alpha/beta hydrolase-like protein). In the homologous recombination vector, the altered portion of the alpha/beta hydrolase-like gene is flanked at its 5N and 3N ends by additional nucleic acid of the alpha/beta hydrolase-like gene to allow for homologous recombination to occur between the exogenous alpha/beta hydrolase-like gene carried by the vector and an endogenous alpha/beta hydrolase-like gene in an embryonic stem cell. The additional flanking alpha/beta hydrolase-like nucleic acid is of sufficient length for successful homologous recombination with the endogenous gene. Typically, several kilobases of flanking DNA (at both the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation), and cells in which the introduced alpha/beta hydrolase-like gene has homologously recombined with the endogenous alpha/beta hydrolase-like gene are selected (see, e.g., Li et al. (1992) Cell 69:915). The selected cells are then injected into a blastocyst of an animal (e.g., a mouse) to form aggregation chimeras (see, e.g., Bradley (1987) in Teratocarcinomas and Embryonic Stem Cells: A Practical Approach, ed. Robertson (IRL, Oxford pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic nonhuman animals containing selected systems that allow for regulated expression of the transgene can be produced. One example of such a system is the cre/loxP recombinase system of bacteriophage PI. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut et al. (1997) Nature 385:810-813 and PCT Publication Nos. WO 97/07668 and WO 97/07669.

IV. Pharmaceutical Compositions

The alpha/beta hydrolase-like nucleic acid molecules, alpha/beta hydrolase-like proteins, and anti-alpha/beta hydrolase-like antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Antibodies, made to any of the polypeptides comprising SEQ ID NO:2 or made to any of the allelic variants or fragments of SEQ ID NO:2, can be used to treat disorders involving the breast, lung, brain, colon, and ovary. More specifically, such antibodies can be used to treat and/or diagnose breast and lung cancer.

The compositions of the invention are useful to treat any of the disorders discussed herein. The compositions are provided in therapeutically effective amounts. By “therapeutically effective amounts” is intended an amount sufficient to modulate the desired response. As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The small molecule can be useful for treating cancer, more particularly breast and lung cancer. The small molecule can be selected from a group consisting of peptides, peptidomimetics and polynucleotides. The small molecule will preferably have a molecular weight less than 10,000 grams per mole.

It is understood that appropriate doses of small molecule agents depends upon a number of factors within the knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL θ (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound (e.g., an alpha/beta hydrolase-like protein or anti-alpha/beta hydrolase-like antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. Depending on the type and severity of the disease, about 1 μg/kg to about 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: (a) screening assays; (b) detection assays (e.g., chromosomal mapping, tissue typing, forensic biology); (c) predictive medicine (e.g., diagnostic assays, prognostic assays, monitoring clinical trials, and pharmacogenomics); and (d) methods of treatment (e.g., therapeutic and prophylactic). The isolated nucleic acid molecules of the invention can be used to express alpha/beta hydrolase-like protein (e.g., via a recombinant expression vector in a host cell in gene therapy applications), to detect alpha/beta hydrolase-like mRNA (e.g., in a biological sample) or a genetic lesion in an alpha/beta hydrolase-like gene, and to modulate alpha/beta hydrolase-like activity. In addition, the alpha/beta hydrolase-like proteins can be used to screen drugs or compounds that are involved in lipid and cholesterol metabolism, in neurotransmission, in regulation of the cell cycle, growth and differentiation, as well as to treat disorders characterized by insufficient or excessive production of alpha/beta hydrolase-like protein or production of alpha/beta hydrolase-like protein forms that have decreased or aberrant activity compared to alpha/beta hydrolase-like wild type protein. In addition, the anti-alpha/beta hydrolase-like antibodies of the invention can be used to detect and isolate alpha/beta hydrolase-like proteins and modulate alpha/beta hydrolase-like activity.

A. Screening Assays

The invention provides a method (also referred to herein as a “screening assay”) for identifying modulators, i.e., candidate or test compounds or agents (e.g., peptides, peptidomimetics, small molecules, or other drugs) that bind to alpha/beta hydrolase-like proteins or have a stimulatory or inhibitory effect on, for example, alpha/beta hydrolase-like expression or alpha/beta hydrolase-like activity.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

Determining the ability of the test compound to bind to the alpha/beta hydrolase-like protein can be accomplished, for example, by coupling the test compound with a radioisotope or enzymatic label such that binding of the test compound to the alpha/beta hydrolase-like protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In a similar manner, one may determine the ability of the alpha/beta hydrolase-like protein to bind to or interact with an alpha/beta hydrolase-like target molecule. By “target molecule” is intended a molecule with which an alpha/beta hydrolase-like protein binds or interacts in nature. In a preferred embodiment, the ability of the alpha/beta hydrolase-like protein to bind to or interact with an alpha/beta hydrolase-like target molecule can be determined by monitoring the activity of the target molecule. For example, the activity of the target molecule can be monitored by detecting.

In yet another embodiment, an assay of the present invention is a cell-free assay comprising contacting an alpha/beta hydrolase-like protein or biologically active portion thereof with a test compound and determining the ability of the test compound to bind to the alpha/beta hydrolase-like protein or biologically active portion thereof. Binding of the test compound to the alpha/beta hydrolase-like protein can be determined either directly or indirectly as described above. In a preferred embodiment, the assay includes contacting the alpha/beta hydrolase-like protein or biologically active portion thereof with a known compound that binds alpha/beta hydrolase-like protein to form an assay mixture, contacting the assay mixture with a test compound, and determining the ability of the test compound to preferentially bind to alpha/beta hydrolase-like protein or biologically active portion thereof as compared to the known compound.

In another embodiment, an assay is a cell-free assay comprising contacting alpha/beta hydrolase-like protein or biologically active portion thereof with a test compound and determining the ability of the test compound to modulate (e.g., stimulate or inhibit) the activity of the alpha/beta hydrolase-like protein or biologically active portion thereof. Determining the ability of the test compound to modulate the activity of an alpha/beta hydrolase-like protein can be accomplished, for example, by determining the ability of the alpha/beta hydrolase-like protein to bind to an alpha/beta hydrolase-like target molecule as described above for determining direct binding. In an alternative embodiment, determining the ability of the test compound to modulate the activity of an alpha/beta hydrolase-like protein can be accomplished by determining the ability of the alpha/beta hydrolase-like protein to further modulate an alpha/beta hydrolase-like target molecule. For example, the catalytic/enzymatic activity of the target molecule on an appropriate substrate can be determined as previously described.

In yet another embodiment, the cell-free assay comprises contacting the alpha/beta hydrolase-like protein or biologically active portion thereof with a known compound that binds an alpha/beta hydrolase-like protein to form an assay mixture, contacting the assay mixture with a test compound, and determining the ability of the test compound to preferentially bind to or modulate the activity of an alpha/beta hydrolase-like target molecule.

In the above-mentioned assays, it may be desirable to immobilize either an alpha/beta hydrolase-like protein or its target molecule to facilitate separation of complexed from uncomplexed forms of one or both of the proteins, as well as to accommodate automation of the assay. In one embodiment, a fusion protein can be provided that adds a domain that allows one or both of the proteins to be bound to a matrix. For example, glutathione-S-transferase/alpha/beta hydrolase-like fusion proteins or glutathione-S-transferase/target fusion proteins can be adsorbed onto glutathione sepharose beads (Sigma Chemical, St. Louis, Mo.) or glutathione-derivatized microtitre plates, which are then combined with the test compound or the test compound and either the nonadsorbed target protein or alpha/beta hydrolase-like protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of alpha/beta hydrolase-like binding or activity determined using standard techniques.

Other techniques for immobilizing proteins on matrices can also be used in the screening assays of the invention. For example, either alpha/beta hydrolase-like protein or its target molecule can be immobilized utilizing conjugation of biotin and streptavidin. Biotinylated alpha/beta hydrolase-like molecules or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with an alpha/beta hydrolase-like protein or target molecules but which do not interfere with binding of the alpha/beta hydrolase-like protein to its target molecule can be derivatized to the wells of the plate, and unbound target or alpha/beta hydrolase-like protein trapped in the wells by antibody conjugation. Methods for detecting such complexes, in addition to those described above for the GST-immobilized complexes, include immunodetection of complexes using antibodies reactive with the alpha/beta hydrolase-like protein or target molecule, as well as enzyme-linked assays that rely on detecting an enzymatic activity associated with the alpha/beta hydrolase-like protein or target molecule.

In another embodiment, modulators of alpha/beta hydrolase-like expression are identified in a method in which a cell is contacted with a candidate compound and the expression of alpha/beta hydrolase-like mRNA or protein in the cell is determined relative to expression of alpha/beta hydrolase-like mRNA or protein in a cell in the absence of the candidate compound. When expression is greater (statistically significantly greater) in the presence of the candidate compound than in its absence, the candidate compound is identified as a stimulator of alpha/beta hydrolase-like mRNA or protein expression. Alternatively, when expression is less (statistically significantly less) in the presence of the candidate compound than in its absence, the candidate compound is identified as an inhibitor of alpha/beta hydrolase-like mRNA or protein expression. The level of alpha/beta hydrolase-like mRNA or protein expression in the cells can be determined by methods described herein for detecting alpha/beta hydrolase-like mRNA or protein.

In yet another aspect of the invention, the alpha/beta hydrolase-like proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with alpha/beta hydrolase-like protein (“alpha/beta hydrolase-like-binding proteins” or “alpha/beta hydrolase-like-bp”) and modulate alpha/beta hydrolase-like activity. Such alpha/beta hydrolase-like-binding proteins are also likely to be involved in the propagation of signals by the alpha/beta hydrolase-like proteins as, for example, upstream or downstream elements of the alpha/beta hydrolase-like pathway.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (1) map their respective genes on a chromosome; (2) identify an individual from a minute biological sample (tissue typing); and (3) aid in forensic identification of a biological sample. These applications are described in the subsections below.

1. Chromosome Mapping

The isolated complete or partial alpha/beta hydrolase-like gene sequences of the invention can be used to map their respective alpha/beta hydrolase-like genes on a chromosome, thereby facilitating the location of gene regions associated with genetic disease. Computer analysis of alpha/beta hydrolase-like sequences can be used to rapidly select PCR primers (preferably 15-25 bp in length) that do not span more than one exon in the genomic DNA, thereby simplifying the amplification process. These primers can then be used for PCR screening of somatic cell hybrids containing individual human chromosomes. Only those hybrids containing the human gene corresponding to the alpha/beta hydrolase-like sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow (because they lack a particular enzyme), but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes (D'Eustachio et al. (1983) Science 220:919-924). Somatic cell hybrids containing only fragments of human chromosomes can also be produced by using human chromosomes with translocations and deletions.

Other mapping strategies that can similarly be used to map an alpha/beta hydrolase-like sequence to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Furthermore, fluorescence in situ hybridization (FISH) of a DNA sequence to a metaphase chromosomal spread can be used to provide a precise chromosomal location in one step. For a review of this technique, see Verma eta a. (1988) Human Chromosomes: A Manual of Basic Techniques (Pergamon Press, NY). The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results in a reasonable amount of time.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Another strategy to map the chromosomal location of alpha/beta hydrolase-like genes uses alpha/beta hydrolase-like polypeptides and fragments and sequences of the present invention and antibodies specific thereto. This mapping can be carried out by specifically detecting the presence of a alpha/beta hydrolase-like polypeptide in members of a panel of somatic cell hybrids between cells of a first species of animal from which the protein originates and cells from a second species of animal, and then determining which somatic cell hybrid(s) expresses the polypeptide and noting the chromosomes(s) from the first species of animal that it contains. For examples of this technique, see Pajunen et al. (1988) Cytogenet. Cell Genet. 47:37-41 and Van Keuren et al. (1986) Hum. Genet. 74:34-40. Alternatively, the presence of a alpha/beta hydrolase-like polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with the alpha/beta hydrolase-like gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The alpha/beta hydrolase-like sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes and probed on a Southern blot to yield unique bands for identification. The sequences of the present invention are useful as additional DNA markers for RFLP (described, e.g., in U.S. Pat. No. 5,272,057).

Furthermore, the sequences of the present invention can be used to provide an alternative technique for determining the actual base-by-base DNA sequence of selected portions of an individual's genome. Thus, the alpha/beta hydrolase-like sequences of the invention can be used to prepare two PCR primers from the 5N and 3N ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The alpha/beta hydrolase-like sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. The noncoding sequences of SEQ ID NO:1 can comfortably provide positive individual identification with a panel of perhaps 10 to 1,000 primers that each yield a noncoding amplified sequence of 100 bases. If a predicted coding sequence, such as that in SEQ ID NO:2, is used, a more appropriate number of primers for positive individual identification would be 500 to 2,000.

3. Use of Partial Alpha/Beta Hydrolase-Like Sequences in Forensic Biology

DNA-based identification techniques can also be used in forensic biology. In this manner, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” that is unique to a particular individual. As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the alpha/beta hydrolase-like sequences or portions thereof, e.g., fragments derived from the noncoding regions of SEQ ID NO:1 having a length of at least 20 or 30 bases.

The alpha/beta hydrolase-like sequences described herein can further be used to provide polynucleotide reagents, e.g., labeled or labelable probes that can be used in, for example, an in situ hybridization technique, to identify a specific tissue. This can be very useful in cases where a forensic pathologist is presented with a tissue of unknown origin. Panels of such alpha/beta hydrolase-like probes, can be used to identify tissue by species and/or by organ type.

In a similar fashion, these reagents, e.g., alpha/beta hydrolase-like primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. These applications are described in the subsections below.

1. Diagnostic Assays

One aspect of the present invention relates to diagnostic assays for detecting alpha/beta hydrolase-like protein and/or nucleic acid expression as well as alpha/beta hydrolase-like activity, in the context of a biological sample. An exemplary method for detecting the presence or absence of alpha/beta hydrolase-like proteins in a biological sample involves obtaining a biological sample from a test subject and contacting the biological sample with a compound or an agent capable of detecting alpha/beta hydrolase-like protein or nucleic acid (e.g., mRNA, genomic DNA) that encodes alpha/beta hydrolase-like protein such that the presence of alpha/beta hydrolase-like protein is detected in the biological sample. Results obtained with a biological sample from the test subject may be compared to results obtained with a biological sample from a control subject.

A preferred agent for detecting alpha/beta hydrolase-like mRNA or genomic DNA is a labeled nucleic acid probe capable of hybridizing to alpha/beta hydrolase-like mRNA or genomic DNA. The nucleic acid probe can be, for example, a full-length alpha/beta hydrolase-like nucleic acid, such as the nucleic acid of SEQ ID NO:1, or a portion thereof, such as a nucleic acid molecule of at least 15, 30, 50, 100, 250, or 500 nucleotides in length and sufficient to specifically hybridize under stringent conditions to alpha/beta hydrolase-like mRNA or genomic DNA. Other suitable probes for use in the diagnostic assays of the invention are described herein.

A preferred agent for detecting alpha/beta hydrolase-like protein is an antibody capable of binding to alpha/beta hydrolase-like protein, preferably an antibody with a detectable label. Antibodies can be polyclonal, or more preferably, monoclonal. An intact antibody, or a fragment thereof (e.g., Fab or F(abN)₂)can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect alpha/beta hydrolase-like mRNA, protein, or genomic DNA in a biological sample in vitro as well as in vivo. For example, in vitro techniques for detection of alpha/beta hydrolase-like mRNA include Northern hybridizations and in situ hybridizations. In vitro techniques for detection of alpha/beta hydrolase-like protein include enzyme linked immunosorbent assays (ELISAs), Western blots, immunoprecipitations, and immunofluorescence. In vitro techniques for detection of alpha/beta hydrolase-like genomic DNA include Southern hybridizations. Furthermore, in vivo techniques for detection of alpha/beta hydrolase-like protein include introducing into a subject a labeled anti-alpha/beta hydrolase-like antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject.

The invention also encompasses kits for detecting the presence of alpha/beta hydrolase-like proteins in a biological sample (a test sample). Such kits can be used to determine if a subject is suffering from or is at increased risk of developing a disorder associated with aberrant expression of alpha/beta hydrolase-like protein (e.g., a hyperproliferative and/or neurodegenerative disorder). For example, the kit can comprise a labeled compound or agent capable of detecting alpha/beta hydrolase-like protein or mRNA in a biological sample and means for determining the amount of an alpha/beta hydrolase-like protein in the sample (e.g., an anti-alpha/beta hydrolase-like antibody or an oligonucleotide probe that binds to DNA encoding an alpha/beta hydrolase-like protein, e.g., SEQ ID NO:1). Kits can also include instructions for observing that the tested subject is suffering from or is at risk of developing a disorder associated with aberrant expression of alpha/beta hydrolase-like sequences if the amount of alpha/beta hydrolase-like protein or mRNA is above or below a normal level.

For antibody-based kits, the kit can comprise, for example: (1) a first antibody (e.g., attached to a solid support) that binds to alpha/beta hydrolase-like protein; and, optionally, (2) a second, different antibody that binds to alpha/beta hydrolase-like protein or the first antibody and is conjugated to a detectable agent. For oligonucleotide-based kits, the kit can comprise, for example: (1) an oligonucleotide, e.g., a detectably labeled oligonucleotide, that hybridizes to an alpha/beta hydrolase-like nucleic acid sequence or (2) a pair of primers useful for amplifying an alpha/beta hydrolase-like nucleic acid molecule.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for observing whether the tested subject is suffering from or is at risk of developing a disorder associated with aberrant expression of alpha/beta hydrolase-like proteins.

2. Other Diagnostic Assays

In another aspect, the invention features a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a alpha/beta hydrolase-like nucleic acid, preferably purified, polypeptide, preferably purified, or antibody, and thereby evaluating the plurality of capture probes. Binding, e.g., in the case of a nucleic acid, hybridization, with a capture probe at an address of the plurality, is detected, e.g., by signal generated from a label attached to the alpha/beta hydrolase-like nucleic acid, polypeptide, or antibody. The capture probes can be a set of nucleic acids from a selected sample, e.g., a sample of nucleic acids derived from a control or non-stimulated tissue or cell.

The method can include contacting the alpha/beta hydrolase-like nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of a alpha/beta hydrolase-like sequence of the invention. Such methods can be used to diagnose a subject, e.g., to evaluate risk for a disease or disorder, to evaluate suitability of a selected treatment for a subject, to evaluate whether a subject has a disease or disorder. Thus, for example, the 33166 sequence set forth in SEQ ID NO:1 encodes a alpha/beta hydrolase-like polypeptide that is associated with an ABH activity.

The method can be used to detect single nucleotide polymorphisms (SNPs), as described below.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express a alpha/beta hydrolase-like polypeptide of the invention or from a cell or subject in which a alpha/beta hydrolase-like-mediated response has been elicited, e.g., by contact of the cell with a alpha/beta hydrolase-like nucleic acid or protein of the invention, or administration to the cell or subject a alpha/beta hydrolase-like nucleic acid or protein of the invention; contacting the array with one or more inquiry probes, wherein an inquiry probe can be a nucleic acid, polypeptide, or antibody (which is preferably other than a alpha/beta hydrolase-like nucleic acid, polypeptide, or antibody of the invention); providing a two dimensional array having a plurality of addresses, each address of the plurality being positionally distinguishable from each other address of the plurality, and each address of the plurality having a unique capture probe, e.g., wherein the capture probes are from a cell or subject which does not express a alpha/beta hydrolase-like sequence of the invention (or does not express as highly as in the case of the alpha/beta hydrolase-like positive plurality of capture probes) or from a cell or subject in which a alpha/beta hydrolase-like-mediated response has not been elicited (or has been elicited to a lesser extent than in the first sample); contacting the array with one or more inquiry probes (which is preferably other than a alpha/beta hydrolase-like nucleic acid, polypeptide, or antibody of the invention), and thereby evaluating the plurality of capture probes. Binding, e.g., in the case of a nucleic acid, hybridization, with a capture probe at an address of the plurality, is detected, e.g., by signal generated from a label attached to the nucleic acid, polypeptide, or antibody.

In another aspect, the invention features a method of analyzing a alpha/beta hydrolase-like sequence of the invention, e.g., analyzing structure, function, or relatedness to other nucleic acid or amino acid sequences. The method includes: providing a alpha/beta hydrolase-like nucleic acid or amino acid sequence, e.g., the 33166 sequence set forth in SEQ ID NO:1 or a portion thereof; comparing the alpha/beta hydrolase-like sequence with one or more preferably a plurality of sequences from a collection of sequences, e.g., a nucleic acid or protein sequence database; to thereby analyze the alpha/beta hydrolase-like sequence of the invention.

The method can include evaluating the sequence identity between a alpha/beta hydrolase-like sequence of the invention, e.g., the 33166 sequence, and a database sequence. The method can be performed by accessing the database at a second site, e.g., over the internet.

In another aspect, the invention features, a set of oligonucleotides, useful, e.g., for identifying SNP's, or identifying specific alleles of a alpha/beta hydrolase-like sequence of the invention, e.g., the 33166 sequence. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotides which hybridizes to one allele provides a signal that is distinguishable from an oligonucleotides which hybridizes to a second allele.

3. Prognostic Assays

The methods described herein can furthermore be utilized as diagnostic or prognostic assays to identify subjects having or at risk of developing a disease or disorder associated with alpha/beta hydrolase-like protein, alpha/beta hydrolase-like nucleic acid expression, or alpha/beta hydrolase-like activity. Prognostic assays can be used for prognostic or predictive purposes to thereby prophylactically treat an individual prior to the onset of a disorder characterized by or associated with alpha/beta hydrolase-like protein, alpha/beta hydrolase-like nucleic acid expression, or alpha/beta hydrolase-like activity.

Thus, the present invention provides a method in which a test sample is obtained from a subject, and alpha/beta hydrolase-like protein or nucleic acid (e.g., mRNA, genomic DNA) is detected, wherein the presence of alpha/beta hydrolase-like protein or nucleic acid is diagnostic for a subject having or at risk of developing a disease or disorder associated with aberrant alpha/beta hydrolase-like expression or activity. As used herein, a “test sample” refers to a biological sample obtained from a subject of interest. For example, a test sample can be a biological fluid (e.g., serum), cell sample, or tissue.

Furthermore, using the prognostic assays described herein, the present invention provides methods for determining whether a subject can be administered a specific agent (e.g., an agonist, antagonist, peptidomimetic, protein, peptide, nucleic acid, small molecule, or other drug candidate) or class of agents (e.g., agents of a type that decrease alpha/beta hydrolase-like activity) to effectively treat a disease or disorder associated with aberrant alpha/beta hydrolase-like expression or activity. In this manner, a test sample is obtained and alpha/beta hydrolase-like protein or nucleic acid is detected. The presence of alpha/beta hydrolase-like protein or nucleic acid is diagnostic for a subject that can be administered the agent to treat a disorder associated with aberrant alpha/beta hydrolase-like expression or activity.

The methods of the invention can also be used to detect genetic lesions or mutations in an alpha/beta hydrolase-like gene, thereby determining if a subject with the lesioned gene is at risk for a disorder characterized by aberrant cell proliferation and/or differentiation or aberrant ABH activity. In preferred embodiments, the methods include detecting, in a sample of cells from the subject, the presence or absence of a genetic lesion or mutation characterized by at least one of an alteration affecting the integrity of a gene encoding an alpha/beta hydrolase-like-protein, or the misexpression of the alpha/beta hydrolase-like gene. For example, such genetic lesions or mutations can be detected by ascertaining the existence of at least one of: (1) a deletion of one or more nucleotides from an alpha/beta hydrolase-like gene; (2) an addition of one or more nucleotides to an alpha/beta hydrolase-like gene; (3) a substitution of one or more nucleotides of an alpha/beta hydrolase-like gene; (4) a chromosomal rearrangement of an alpha/beta hydrolase-like gene; (5) an alteration in the level of a messenger RNA transcript of an alpha/beta hydrolase-like gene; (6) an aberrant modification of an alpha/beta hydrolase-like gene, such as of the methylation pattern of the genomic DNA; (7) the presence of a non-wild-type splicing pattern of a messenger RNA transcript of an alpha/beta hydrolase-like gene; (8) a non-wild-type level of an alpha/beta hydrolase-like-protein; (9) an allelic loss of an alpha/beta hydrolase-like gene; and (10) an inappropriate post-translational modification of an alpha/beta hydrolase-like-protein. As described herein, there are a large number of assay techniques known in the art that can be used for detecting lesions in an alpha/beta hydrolase-like gene. Any cell type or tissue, preferably peripheral blood leukocytes, in which alpha/beta hydrolase-like proteins are expressed may be utilized in the prognostic assays described herein.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the alpha/beta hydrolase-like-gene (see, e.g., Abravaya et al. (1995) Nucleic Acids Res. 23:675-682). It is anticipated that PCR and/or LCR may be desirable to use as a preliminary amplification step in conjunction with any of the techniques used for detecting mutations described herein.

Alternative amplification methods include self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an alpha/beta hydrolase-like gene from a sample cell can be identified by alterations in restriction enzyme cleavage patterns of isolated test sample and control DNA digested with one or more restriction endonucleases. Moreover, the use of sequence specific ribozymes (see, e.g., U.S. Pat. No. 5,498,531) can be used to score for the presence of specific mutations by development or loss of a ribozyme cleavage site.

A method for treating a disorder involving breast and lung cancer would comprise administering a ribozyme that has a complementary region to an mRNA transcript and is capable of cleaving said transcript wherein said transcript is encoded by the polynucleotide sequence shown in SEQ ID NO:1.

In other embodiments, genetic mutations in an alpha/beta hydrolase-like molecule can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). In yet another embodiment, any of a variety of sequencing reactions known in the art can be used to directly sequence the alpha/beta hydrolase-like gene and detect mutations by comparing the sequence of the sample alpha/beta hydrolase-like gene with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the alpha/beta hydrolase-like gene include methods in which protection from cleavage agents is used to detect mismatched bases in RNA/RNA or RNA/DNA heteroduplexes (Myers et al. (1985) Science 230:1242). See, also Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more “DNA mismatch repair” enzymes that recognize mismatched base pairs in double-stranded DNA in defined systems for detecting and mapping point mutations in alpha/beta hydrolase-like cDNAs obtained from samples of cells. See, e.g., Hsu et al. (1994) Carcinogenesis 15:1657-1662. According to an exemplary embodiment, a probe based on an alpha/beta hydrolase-like sequence, e.g., a wild-type alpha/beta hydrolase-like sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in alpha/beta hydrolase-like genes. For example, single-strand conformation polymorphism (SSCP) may be used to detect differences in electrophoretic mobility between mutant and wild-type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc. Natl. Acad. Sci. USA 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology, which depends on selective PCR amplification, may be used in conjunction with the instant invention. Oligonucleotides used as primers for specific amplification may carry the mutation of interest in the center of the molecule so that amplification depends on differential hybridization (Gibbs et al. (1989) Nucleic Acids Res. 17:2437-2448) or at the extreme 3N end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3N end of the 5N sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnosed patients exhibiting symptoms or family history of a disease or illness involving an alpha/beta hydrolase-like gene.

4. Pharmacogenomics

Agents, or modulators that have a stimulatory or inhibitory effect on alpha/beta hydrolase-like activity (e.g., alpha/beta hydrolase-like gene expression) as identified by a screening assay described herein, can be administered to individuals to treat (prophylactically or therapeutically) disorders associated with aberrant alpha/beta hydrolase-like activity as well as to modulate the phenotype of cellular and physiological processes associated with this activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of alpha/beta hydrolase-like protein, expression of alpha/beta hydrolase-like nucleic acid, or mutation content of alpha/beta hydrolase-like genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2): 254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (antimalarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, an “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a alpha/beta hydrolase-like protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a alpha/beta hydrolase-like molecule or alpha/beta hydrolase-like modulator of the present invention) can give an indication whether gene pathways related to toxicity have been turned on.

Information generated from more than one of the above phannacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a alpha/beta hydrolase-like molecule or alpha/beta hydrolase-like modulator of the invention, such as a modulator identified by one of the exemplary screening assays described herein.

The present invention further provides methods for identifying new agents, or combinations, that are based on identifying agents that modulate the activity of one or more of the gene products encoded by one or more of the alpha/beta hydrolase-like genes of the present invention, wherein these products may be associated with resistance of the cells to a therapeutic agent. Specifically, the activity of the proteins encoded by the alpha/beta hydrolase-like genes of the present invention can be used as a basis for identifying agents for overcoming agent resistance. By blocking the activity of one or more of the resistance proteins, target cells will become sensitive to treatment with an agent that the unmodified target cells were resistant to.

Monitoring the influence of agents (e.g., drugs) on the expression or activity of a alpha/beta hydrolase-like protein can be applied in clinical trials. For example, the effectiveness of an agent determined by a screening assay as described herein to increase alpha/beta hydrolase-like gene expression, protein levels, or upregulate alpha/beta hydrolase-like activity, can be monitored in clinical trials of subjects exhibiting decreased alpha/beta hydrolase-like gene expression, protein levels, or downregulated alpha/beta hydrolase-like activity. Alternatively, the effectiveness of an agent determined by a screening assay to decrease alpha/beta hydrolase-like gene expression, protein levels, or downregulate alpha/beta hydrolase-like activity, can be monitored in clinical trials of subjects exhibiting increased alpha/beta hydrolase-like gene expression, protein levels, or upregulated alpha/beta hydrolase-like activity. In such clinical trials, the expression or activity of a alpha/beta hydrolase-like gene, and preferably, other genes that have been implicated in, for example, a alpha/beta hydrolase-like-associated disorder can be used as a “read out” or markers of the phenotype of a particular cell.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of alpha/beta hydrolase-like protein, expression of alpha/beta hydrolase-like nucleic acid, or mutation content of alpha/beta hydrolase-like genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an alpha/beta hydrolase-like modulator, such as a modulator identified by one of the exemplary screening assays described herein.

5. Monitoring of Effects During Clinical Trials

Monitoring the influence of agents (e.g., drugs, compounds) on the expression or activity of alpha/beta hydrolase-like genes (e.g., the ability to modulate aberrant lipid and cholesterol metabolism; biotransformation of drugs and other chemicals; detoxification; neurotransmission; and cellular cycle regulation, growth and differentiation) can be applied not only in basic drug screening but also in clinical trials. For example, the effectiveness of an agent, as determined by a screening assay as described herein, to increase or decrease alpha/beta hydrolase-like gene expression, protein levels, or protein activity, can be monitored in clinical trials of subjects exhibiting decreased or increased alpha/beta hydrolase-like gene expression, protein levels, or protein activity. In such clinical trials, alpha/beta hydrolase-like expression or activity and preferably that of other genes that have been implicated in for example, a cholesterol and/or lipid metabolism disorder or other ABH-associated disorder can be used as a marker of the responsiveness of a particular cell.

For example, and not by way of limitation, genes that are modulated in cells by treatment with an agent (e.g., compound, drug, or small molecule) that modulates alpha/beta hydrolase-like activity (e.g., as identified in a screening assay described herein) can be identified. Thus, to study the effect of agents on ABH-associated disorders, lipid and/or cholesterol metabolism disorders, for example, in a clinical trial, cells can be isolated and RNA prepared and analyzed for the levels of expression of alpha/beta hydrolase-like genes and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of alpha/beta hydrolase-like genes or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (1) obtaining a preadministration sample from a subject prior to administration of the agent; (2) detecting the level of expression of an alpha/beta hydrolase-like protein, mRNA, or genomic DNA in the preadministration sample; (3) obtaining one or more postadministration samples from the subject; (4) detecting the level of expression or activity of the alpha/beta hydrolase-like protein, mRNA, or genomic DNA in the postadministration samples; (5) comparing the level of expression or activity of the alpha/beta hydrolase-like protein, mRNA, or genomic DNA in the preadministration sample with the alpha/beta hydrolase-like protein, mRNA, or genomic DNA in the postadministration sample or samples; and (vi) altering the administration of the agent to the subject accordingly to bring about the desired effect, i.e., for example, an increase or a decrease in the expression or activity of an alpha/beta hydrolase-like protein.

C. Methods of Treatment

The present invention provides for both prophylactic and therapeutic methods of treating a subject at risk of (or susceptible to) a disorder or having a disorder associated with aberrant alpha/beta hydrolase-like expression or activity. Additionally, the compositions of the invention find use in the treatment of disorders described herein. Thus, therapies for disorders associated with CCC are encompassed herein.

1. Prophylactic Methods

In one aspect, the invention provides a method for preventing in a subject a disease or condition associated with an aberrant alpha/beta hydrolase-like expression or activity by administering to the subject an agent that modulates alpha/beta hydrolase-like expression or at least one alpha/beta hydrolase-like gene activity. Subjects at risk for a disease that is caused, or contributed to, by aberrant alpha/beta hydrolase-like expression or activity can be identified by, for example, any or a combination of diagnostic or prognostic assays as described herein. Administration of a prophylactic agent can occur prior to the manifestation of symptoms characteristic of the alpha/beta hydrolase-like aberrancy, such that a disease or disorder is prevented or, alternatively, delayed in its progression. Depending on the type of alpha/beta hydrolase-like aberrancy, for example, an alpha/beta hydrolase-like agonist or alpha/beta hydrolase-like antagonist agent can be used for treating the subject. The appropriate agent can be determined based on screening assays described herein.

2. Therapeutic Methods

Another aspect of the invention pertains to methods of modulating alpha/beta hydrolase-like expression or activity for therapeutic purposes. The modulatory method of the invention involves contacting a cell with an agent that modulates one or more of the activities of alpha/beta hydrolase-like protein activity associated with the cell. An agent that modulates alpha/beta hydrolase-like protein activity can be an agent as described herein, such as a nucleic acid or a protein, a naturally-occurring cognate ligand of an alpha/beta hydrolase-like protein, a peptide, an alpha/beta hydrolase-like peptidomimetic, or other small molecule. In one embodiment, the agent stimulates one or more of the biological activities of alpha/beta hydrolase-like protein. Examples of such stimulatory agents include active alpha/beta hydrolase-like protein and a nucleic acid molecule encoding an alpha/beta hydrolase-like protein that has been introduced into the cell. In another embodiment, the agent inhibits one or more of the biological activities of alpha/beta hydrolase-like protein. Examples of such inhibitory agents include antisense alpha/beta hydrolase-like nucleic acid molecules and anti-alpha/beta hydrolase-like antibodies. Such agents can be particularly useful for the treatment and diagnosis of breast and lung carcinoma.

These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of an alpha/beta hydrolase-like protein or nucleic acid molecule. In one embodiment, the method involves administering an agent (e.g., an agent identified by a screening assay described herein), or a combination of agents, that modulates (e.g., upregulates or downregulates) alpha/beta hydrolase-like expression or activity. In another embodiment, the method involves administering an alpha/beta hydrolase-like protein or nucleic acid molecule as therapy to compensate for reduced or aberrant alpha/beta hydrolase-like expression or activity.

Stimulation of alpha/beta hydrolase-like activity is desirable in situations in which an alpha/beta hydrolase-like protein is abnormally downregulated and/or in which increased alpha/beta hydrolase-like activity is likely to have a beneficial effect. Conversely, inhibition of alpha/beta hydrolase-like activity is desirable in situations in which alpha/beta hydrolase-like activity is abnormally upregulated and/or in which decreased alpha/beta hydrolase-like activity is likely to have a beneficial effect.

This invention is further illustrated by the following examples, which should not be construed as limiting.

### EXPERIMENTAL

### EXAMPLE 1

### Isolation of 33166

Poly-A+ RNA from primary human osteoblasts were converted to used to generate a cDNA library. EST sequencing was performed on this library, and greater than 10,000 sequences were subjected to database analysis together with other proprietary sequences.

From this analysis, overlapping sequences were combined into a single contiguous sequence. Upon further analysis, the clone 33166 was identified. Clone 33166 encodes an approximately 2.1 Kb mRNA transcript having the corresponding cDNA set forth in FIG. 1 (SEQ ID NO:1). This transcript has a 1320 nucleotide open reading frame (nucleotides 176-1495 of SEQ ID NO:1 corresponding to nucleotides designated 1-1320 in FIG. 1), which encodes a 439 amino acid protein (FIG. 1, SEQ ID NO:2) having a molecular weight of approximately 48.2 kDa. HMMER (version 2) analysis also showed that the polypeptide belongs to the ABH fold protein family.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence which is at least 60% identical to the nucleotide sequence of SEQ ID NO:1, the cDNA insert of the plasmid deposited with ATCC as Accession Number ______, or a complement thereof;
b) a nucleic acid molecule comprising a fragment of at least 15 nucleotides of the nucleotide sequence of SEQ ID NO:1, the cDNA insert of the plasmid deposited with ATCC as Accession Number ______, or a complement thereof;
c) a nucleic acid molecule which encodes a polypeptide comprising the amino acid sequence of SEQ ID NO:2, or an amino acid sequence encoded by the cDNA insert of the plasmid deposited with ATCC as Accession Number ______;
d) a nucleic acid molecule which encodes a fragment of a polypeptide comprising the amino acid sequence of SEQ ID NO:2, or an amino acid sequence encoded by the cDNA insert of the plasmid deposited with ATCC as Accession Number ______, wherein the fragment comprises at least 15 contiguous amino acids of SEQ ID NO:2, or the polypeptide encoded by the cDNA insert of the plasmid deposited with ATCC as Accession Number ______; and
e) a nucleic acid molecule which encodes a naturally occurring allelic variant of a polypeptide comprising the amino acid sequence of SEQ ID NO:2, or an amino acid sequence encoded by the cDNA insert of the plasmid deposited with ATCC as Accession Number ______, wherein the nucleic acid molecule hybridizes to a nucleic acid molecule comprising SEQ ID NO:1, or a complement thereof under stringent conditions.

2. The isolated nucleic acid molecule of claim 1, which is selected from the group consisting of:
a) a nucleic acid comprising the nucleotide sequence of SEQ ID NO:1, the cDNA insert of the plasmid deposited with ATCC as Accession Number ______, or a complement thereof; and
b) a nucleic acid molecule which encodes a polypeptide comprising the amino acid sequence of SEQ ID NO:2, or an amino acid sequence encoded by the cDNA insert of the plasmid deposited with ATCC as Accession Number ______.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A nonhuman mammalian host cell containing the nucleic acid molecule of claim 1.

8. An isolated polypeptide selected from the group consisting of:
a) a fragment of a polypeptide comprising the amino acid sequence of SEQ ID NO:2, or an amino acid sequence encoded by the cDNA insert of the plasmid deposited with ATCC as Accession Number ______, wherein the fragment comprises at least 15 contiguous amino acids of SEQ ID NO:2, or an amino acid sequence encoded by the cDNA insert of the plasmid deposited with ATCC as Accession Number ______;
b) a naturally occurring allelic variant of a polypeptide comprising the amino acid sequence of SEQ ID NO:2, or an amino acid sequence encoded by the cDNA insert of the plasmid deposited with ATCC as Accession Number ______, wherein the polypeptide is encoded by a nucleic acid molecule which hybridizes to a nucleic acid molecule comprising SEQ ID NO:1, or a complement thereof under stringent conditions; and
c) a polypeptide which is encoded by a nucleic acid molecule comprising a nucleotide sequence which is at least 45% identical to a nucleic acid comprising the nucleotide sequence of SEQ ID NO:1, or a complement thereof.

9. The isolated polypeptide of claim 8 comprising the amino acid sequence of SEQ ID NO:2, or an amino acid sequence encoded by the cDNA insert of the plasmid deposited with ATCC as Accession Number ______.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO:2, or an amino acid sequence encoded by the cDNA insert of the plasmid deposited with ATCC as Accession Number ______.
b) a polypeptide comprising a fragment of the amino acid sequence of SEQ ID NO:2, or an amino acid sequence encoded by the cDNA insert of the plasmid deposited with ATCC as Accession Number ______, wherein the fragment comprises at least 15 contiguous amino acids of SEQ ID NO:2, or an amino acid sequence encoded by the cDNA insert of the plasmid deposited with ATCC as Accession Number ______; and
c) a naturally occurring allelic variant of a polypeptide comprising the amino acid sequence of SEQ ID NO:2, or an amino acid sequence encoded by the cDNA insert of the plasmid deposited with ATCC as Accession Number ______, wherein the polypeptide is encoded by a nucleic acid molecule which hybridizes to a nucleic acid molecule comprising SEQ ID NO:1, or a complement thereof under stringent conditions; comprising culturing the host cell of claim 5 under conditions in which the nucleic acid molecule is expressed.

13. The method of claim 12 wherein said polypeptide comprises the amino acid sequence of SEQ ID NO:2, or an amino acid sequence encoded by the cDNA insert of the plasmid deposited with ATCC as Accession Number ______.

14. A method for detecting the presence of a polypeptide of claim 8 in a sample, comprising:
a) contacting the sample with a compound which selectively binds to a polypeptide of claim 8; and
b) determining whether the compound binds to the polypeptide in the sample.

15. The method of claim 14, wherein the compound which binds to the polypeptide is an antibody.

16. A kit comprising a compound which selectively binds to a polypeptide of claim 8 and instructions for use.

17. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

18. The method of claim 17, wherein the sample comprises mRNA molecules and is contacted with a nucleic acid probe.

19. A kit comprising a compound which selectively hybridizes to a nucleic acid molecule of claim 1 and instructions for use.

20. A method for identifying a compound which binds to a polypeptide of claim 8 comprising the steps of:
a) contacting a polypeptide, or a cell expressing a polypeptide of claim 8 with a test compound; and
b) determining whether the polypeptide binds to the test compound.

21. The method of claim 20, wherein the binding of the test compound to the polypeptide is detected by a method selected from the group consisting of:
a) detection of binding by direct detecting of test compound/polypeptide binding;
b) detection of binding using a competition binding assay; and
c) detection of binding using an assay for alpha/beta hydrolase-like activity.

22. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

23. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.

24. A method for modulating the level or activity of the nucleotide sequence shown in SEQ ID NO:1, said method comprising contacting said nucleic acid molecule with an agent under conditions that allow the agent to modulate the level or activity of the nucleic acid molecule.

25. The method of claim 24, wherein said modulation is in cells derived from tissue selected from the group consisting of breast, lung, brain, colon, and ovary.

26. The method of claim 25, wherein said modulation is in vivo.

27. The method of claim 26, wherein said modulation is in a patient having a disorder involving breast, lung, brain, colon, and ovary.

28. A method for treating a disorder involving breast, lung, brain, colon, and ovary in a subject in need of such treatment, said method comprising administering any of the polypeptides of claim 8 to said subject in a therapeutically effective amount.

29. A method for treating lung or breast cancer, in a subject in need of such treatment, said method comprising administering any of the polypeptides of claim 8 to said subject in a therapeutically effective amount.

30. A method for treating lung or breast cancer, in a subject in need of such treatment, said method comprising administering any of the nucleotide sequences of claim 1 to said subject in a therapeutically effective amount.

31. A method of treating lung or breast cancer, in a subject in need of such treatment, said method comprising administering an antibody which binds to any of the polypeptides of claim 8 to said subject in a therapeutically effective amount.

32. An antisense oligonucleotide that inhibits the expression of the protein kinase encoded by SEQ ID NO:1.

33. An antisense oligonucleotide that binds to the translation start site for the protein encoded by SEQ ID NO:1.

34. An antisense oligonucleotide consisting of a sequence of at least 10 nucleotides that is complementary to the coding region for the protein kinase encoded by SEQ ID NO:1.

35. A method for treating a disorder involving breast, lung, brain, colon, and ovary, said method comprising: administering an antisense oligonucleotide that inhibits the expression of the protein kinase encoded by SEQ ID NO:1.

36. A ribozyme that has a complementary region to an mRNA transcript and is capable of cleaving said transcript wherein said transcript is encoded by the polynucleotide sequence shown in SEQ ID NO:1.

37. A method for treating a disorder involving breast, lung, brain, colon, and ovary, said method comprising: administering a ribozyme that has a complementary region to an mRNA transcript and is capable of cleaving said transcript wherein said transcript is encoded by the polynucleotide sequence shown in SEQ ID NO:1.

38. A method for treating a disorder involving breast, lung, brain, colon, and ovary in a subject in need of such treatment, said method comprising: administering a small molecule which can modulate expression of the polypeptide encoded by SEQ ID NO:1.

39. The method according to claim 38, wherein said small molecule has a molecular weight less than 10,000 grams per mole.

40. The method according to claim 39, wherein said small molecule is selected from the group consisting of: peptides, peptidomimetics, polynucleotides, and polynucleotide analogs.


SEQUENCE LISTING (2 sequences)

SEQ ID NO: 1 - DNA, 2057 bp, Homo sapiens
  CDS (172)...(1491)
tatagggagtcgacccacgcgtccggccaggggcaggtgcccgcccgcgtagacgcacccggcctgaccccgcgccaccatgtaaacggcgccagcaggcggacgctggcttctccgcctgggacccctccgccccgacccgggccccgcggccctcgatgaggacacaccatgctgaccggggtgaccgacggtatcttctgttgcctgctgggcacgccccccaacgccgtggggccactggagagcgtcgagtccagcgatggctacacctttgtagaggtcaagcccggccgcgtgctgcgggtgaagcatgcaggacccgccccagccgctgccccacctccaccatcatccgcatcctcggatgcagcccagggggacctctccggcttggtccgctgtcagcgccggatcaccgtgtaccgcaatgggcggttgctggtggaaaacctgggccgagcccctcgagccgacctcctacacgggcagaatggctctggggagccgccggccgccctggaggtggagctggcagatccggcgggcagcgatggccgcttggcccccggcagcgcaggcagcggcagcggcagtggcagtggtgggcggcggcggcgagccaggcgccccaagaggaccatccatattgactgtgagaagcgcatcactagctgcaaaggcgcccaggccgacgtggtgctctttttcatccatggtgtcggcggttccctggccatctggaaggagcagctggacttctttgtgcgcctaggctatgaggtggtggctcctgacctggccggccacggggccagctctgcgccccaggtggccgcagcctacaccttctatgcgctggctgaggacatgcgagcaatcttcaagcgctatgccaagaagcgaaatgtgctcattggccattcctacggtgtctctttctgcacattcctggcacatgagtacccagacctagtgcacaaggtgatcatgatcaatggcgggggccctacggcgctggagcccagcttctgctcaatcttcaacatgcccacctgcgtcctgcactgcttgtcgccctgcctggcctggagcttcctcaaggccggcttcgcccgccaaggagccaaggagaagcagctgttaaaggagggcaacgctttcaacgtgtcatccttcgtactccgggccatgatgagcggccagtactggcccgagggcgacgaggtctaccacgccgagctcaccgtgcccgtcctgcttgtccacggcatgcacgataagtttgtgccggtggaggaagaccagcgcatggccgagatcctgctcctggcattcctgaagctcatcgacgagggcagccacatggtgatgctggaatgccctgagacggtcaacacgctgctccacgaattcctgctctgggagcccgagccctcgcccaaggctctaccggagccactgccggcgcctccagaagacaagaagtagccgctgggccggcggggcatcgcttggtgagcacagccgcagcaggaggaggcccgagcctgcgccaggtctgcagcgcagaccacctgggcgggccgttcgctccggtgggcggggccaggtcagggagacgcccccaggctgcctgggcggggcgtggcatccgagggagcccagcggacattccgctctccgcttccgtcccgcggggcccatcggcgttttggggccgcagccgggaccctcacggaagatgaccttgtacagaagctctccctcaccttccccccaacgccacggccaaggcaggccccccaccccgctgtcttccgtgtcagccgtgcttgatcctgggacccacgagccccacagggaccctcgaggccccatcccgttatccgagacccttcctaccccccattcctcggcgctgggagctatttttgcccaagggggggggatgggggggctggcgccaccgaacctgcacatctcaacttgtaactcaataaacagaagtgacaatcggraaaaaaaaaaaaaaaaaaaaaaaaaaaaaaaaaaaa

SEQ ID NO: 2 - protein, 439 aa, Homo sapiens
MLTGVTDGIFCCLLGTPPNAVGPLESVESSDGYTFVEVKPGRVLRVKHAGPAPAAAPPPPSSASSDAAQGDLSGLVRCQRRITVYRNGRLLVENLGRAPRADLLHGQNGSGEPPAALEVELADPAGSDGRLAPGSAGSGSGSGSGGRRRRARRPKRTIHIDCEKRITSCKGAQADVVLFFIHGVGGSLAIWKEQLDFFVRLGYEVVAPDLAGHGASSAPQVAAAYTFYALAEDMRAIFKRYAKKRNVLIGHSYGVSFCTFLAHEYPDLVHKVIMINGGGPTALEPSFCSIFNMPTCVLHCLSPCLAWSFLKAGFARQGAKEKQLLKEGNAFNVSSFVLRAMMSGQYWPEGDEVYHAELTVPVLLVHGMHDKFVPVEEDQRMAEILLLAFLKLIDEGSHMVMLECPETVNTLLHEFLLWEPEPSPKALPEPLPAPPEDKK